(19) United States
(12) Patent Application Publication
(10) Pub. No.: US 2012/0141497 A1
(43) Pub. Date: Jun. 7, 2012

(54) METHODS OF PURIFYING SMALL MODULAR IMMUNOPHARMACEUTICAL PROTEINS

(75) Inventors: Christopher John Gallo, Windham, NH (US); Shujun Sun, Brentwood, NH (US); James Edward Booth, Andover, MA (US); Jason Richard Cormier, Westford, MA (US); Daniel Patrick Lacasse, Leominister, MA (US); Aaron Russell Noyes, Pepperell, MA (US)

(73) Assignee: Wyeth LLC, Madison, NJ (US)

(21) Appl. No.: 13/255,650

(22) PCT Filed: Mar. 11, 2010
(86) PCT No.: PCT/US10/26931

§ 371 Date: Jan. 24, 2012

Related U.S. Application Data
(60) Provisional application No. 61/159,347, filed on Mar. 11, 2009.

(51) Int. Cl. A61K 39/395; C07K 16/28; C07K 1/18; C07K 1/16; C07K 1/22

(52) U.S. Cl. 424/158.1; 530/389.2

(57) ABSTRACT

The present invention provides, among other things, methods of purifying or recovering proteins, in particular, small modular immunopharmaceutical (SMIPs™) proteins, from protein preparations containing high molecular weight (HMW) aggregates and other impurities based on hydroxyapatite chromatography. In some embodiments, the hydroxyapatite chromatography is used in combination with affinity chromatography and/or ion exchange chromatography. In some embodiments, inventive methods according to the invention involve no more than three chromatography steps. The present invention also provides proteins such as SMIPs™ purified according to the invention and pharmaceutical compositions containing the same.

### REFERENCE TO RELATED APPLICATIONS

This application claims the benefit of U.S. Provisional Patent Application No. 61/159,347, filed Mar. 11, 2009, the contents of which are hereby incorporated by reference in their entireties.

### BACKGROUND OF THE INVENTION

Typically, when proteins are produced for pharmaceutical uses, contaminants must be removed from protein preparations before they can be used in diagnostic applications, therapeutic applications, applied cell biology, and functional studies. For example, protein preparations harvested from cultured cells often contain unwanted components, such as high molecular weight (HMW) aggregates of the protein produced by the cells. The high molecular weight aggregates can adversely affect product safety by causing complement activation or anaphylaxis upon administration. Further, aggregates may hinder manufacturing processes by causing decreased product yield, peak broadening, and loss of activity.

Small modular immunopharmaceutical (SMIP™) proteins belong to a relatively new class of pharmaceutical proteins as compared to antibodies and other therapeutic proteins. Therefore, the purification of SMIP™ proteins is particularly challenging due to lack of familiarity with this type of protein. In addition, SMIP™ proteins have a high propensity to aggregate. For example, the percentage of HMW aggregates in cell culture may be as high as 50-60%.

### SUMMARY OF THE INVENTION

The present invention provides, among other things, effective methods of purifying proteins containing HMW aggregates. The present invention encompasses the discovery that small modular immunopharmaceutical proteins can be purified from protein preparations containing high percentage of HMW aggregates (e.g., more than 50-60%) using no more than three chromatography steps. Thus, inventive methods according to the invention reduce the number of column steps resulting in significantly reduced process time and improved product yield. The present invention is particularly useful for purifying small modular immunopharmaceutical proteins. The methods of the invention may also be used to purify other proteins, in particular, those proteins having a propensity to aggregate.

In one aspect, the present invention provides a method of purifying a small modular immunopharmaceutical protein from a protein preparation containing high molecular weight aggregates including a step of subjecting the protein preparation to hydroxyapatite chromatography under an operating condition such that the purified small modular immunopharmaceutical protein contains less than 4% aggregates (e.g., less than 3.5%, 3%, 2.5%, 2%, 1.5%, 1%, 0.8%, 0.6%, 0.5%, 0.4%, 0.2%, or 0.1%). In some embodiments, a method according to the invention involves no more than 3 chromatography steps.

In some embodiments, the operating condition includes eluting the small modular immunopharmaceutical protein from a hydroxyapatite chromatography column in a phosphate buffer. In some embodiments, the phosphate buffer is endotoxin-free. In some embodiments, the phosphate buffer is depyrogenated. In some embodiments, the phosphate buffer comprises sodium phosphate, potassium phosphate, and/or lithium phosphate. In some embodiments, a suitable phosphate buffer contains sodium phosphate at a concentration ranging from 1 mM to 50 mM. In some embodiments, a suitable phosphate buffer further contains sodium chloride at a concentration ranging from 100 mM to 2.5 M. In some embodiments, a suitable phosphate buffer contains sodium phosphate at a concentration ranging from 2 mM to 32 mM and sodium chloride at a concentration ranging from 100 mM to 1.6 M. In some embodiments, a suitable phosphate buffer has a pH ranging from 6.5 to 8.5.

In some embodiments, the operating condition includes eluting the small modular immunopharmaceutical protein from a hydroxyapatite chromatography column by a NaCl gradient. In some embodiments, the operating condition includes eluting the small modular immunopharmaceutical protein from a hydroxyapatite chromatography column by a NaCl step elution method. In some embodiments, the operating condition includes eluting the small modular immunopharmaceutical protein from a hydroxyapatite chromatography column by a phosphate gradient (e.g., a linear phosphate gradient).

In some embodiments, the hydroxyapatite chromatography uses a column containing ceramic hydroxyapatite Type I or Type II resins. In some embodiments, the column contains ceramic hydroxyapatite Type I resins. In some embodiments, the resins suitable for the hydroxyapatite chromatography are 1 μm to 1,000 μm in diameter. In some embodiments, the resins suitable for the hydroxyapatite chromatography are 10 μm to 100 μm in diameter.

In some embodiments, the method further includes a step of purifying the protein preparation by affinity chromatography before the step of hydroxyapatite chromatography. In some embodiments, the affinity chromatography step uses a protein absorbent that binds to a constant immunoglobulin domain. In some embodiments, the affinity chromatography uses a protein absorbent that binds to a variable immunoglobulin domain. In some embodiments, a protein absorbent suitable for the invention binds to a VH₃ domain or a domain homologous to VH₃ (e.g., a domain from the VH₃ family). In some embodiments, a protein absorbent suitable for the invention comprises protein A. In some embodiments, the affinity chromatography step uses a MabSelect™ rProtein A resin column. In some embodiments, a method according to the invention further includes a step of adding an additive (e.g., PEG and/or other nonionic organic polymers) to promote binding to protein sorbents.

In some embodiments, the step of affinity chromatography comprises washing an affinity chromatography column using a washing buffer comprising Hepes, sodium chloride, calcium chloride, arginine, Tris, magnesium chloride, histidine, urea, imidazole, one or more organic solvents (e.g., ethanol, methanol, propylene glycol, ethylene glycol, propanol, isopropanol, and butanol), and/or detergents (e.g., ionic or nonionic). In some embodiments, the step of affinity chromatography comprises eluting the small modular immunopharmaceutical protein from an affinity chromatography column using an elution buffer comprising Hepes, phosphoric acid, glycine, glycylglycine, magnesium chloride, urea, propylene glycol, ethylene glycol, one or more organic acids (e.g., acetic acid, citric acid, formic acid, lactic acid, tartaric acid, malic acid, malonic acid, phthalic acid and salicyclic acid), and/or arginine. In some embodiments, the elution buffer further comprises a salt selected from the group consisting of sodium chloride, potassium chloride, calcium chloride, magnesium chloride, and combinations thereof. In some embodiments, the salt is at a concentration ranging from 1 mM to 1 M. In certain embodiments, the salt is at a concentration ranging from 1 mM to 500 mM. In certain embodiments, the salt is at a concentration ranging from 1 mM to 100 mM.

In some embodiments, a method according to the invention further comprises a step of purifying the protein preparation by anion exchange chromatography using an anion exchange chromatography resin. In certain embodiments, a method according to the invention further comprises a step of purifying the protein preparation by anion exchange chromatography after the affinity chromatography but before the hydroxyapatite chromatography step. In some embodiments, a method according to the invention further comprises a step of adding an additive to enhance binding of the small modular immunopharmaceutical protein and/or impurities to the anion exchange chromatography resin. In some embodiments, the additive added induces precipitation of one or more contaminants or impurities from the protein preparation. In some embodiments, the precipitated contaminants are removed from the protein preparation by filtration. In some embodiments, a suitable additive is or contains a nonionic organic polymer (e.g., polyethylene glycol (PEG), polypropylene glycol, cellulose, dextran, starch, and/or polyvinylpyrrolidone).

In some embodiments, the method further comprises a step of applying the protein preparation to a depth filter before the affinity or anion exchange chromatography.

In some embodiments, the method further comprises one or more filtration steps. In some embodiments, the one or more filtration steps comprise a virus retaining filtration step. In some embodiments, the one or more filtration steps comprise ultrafiltration and/or diafiltration steps.

In some embodiments, the protein preparation is prepared from cultured bacterial cells, mammalian cells, plant cells, yeast cells, insect cells, cell-free medium, transgenic animals or plants. In some embodiments, the protein preparation is a cell culture medium preparation. In some embodiments, the culture medium preparation contains the small modular immunopharmaceutical protein secreted from cultured cells. In certain embodiments, the cultured cells are CHO cells. In certain embodiments, the culture medium preparation is prepared from a large scale bioreactor. In some embodiments, the protein preparation to be purified contains a cell extract. In some embodiments, the protein preparation to be purified is prepared from inclusion bodies.

In another aspect, the present invention provides methods of purifying a small modular immunopharmaceutical protein from a protein preparation containing high molecular weight aggregates by subjecting the protein preparation to (a) affinity chromatography and/or ion exchange chromatography (e.g., one or two ion exchange chromatography steps), and (b) hydroxyapatite chromatography under operating conditions such that the purified small modular immunopharmaceutical protein contains less than 4% (e.g., less than 3.5%, 3%, 2.5%, 2%, 1.5%, 1%, 0.8%, 0.6%, 0.5%, 0.4%, 0.2%, 0.1%) aggregates. In some embodiments, the protein preparation is subjected to (a1) affinity chromatography, (a2) ion exchange chromatography, and (b) hydroxyapatite chromatography. In some embodiments, the protein preparation is subjected to (a1) cation exchange chromatography, (a2) anion exchange chromatography, and (b) hydroxyapatite chromatography. In some embodiments, the affinity chromatography is protein A chromatography. In some embodiments, the ion exchange chromatography is anion or cation exchange chromatography. In some embodiments, the ion exchange chromatography resin is selected from the group consisting of Q Sepharose™ FF, Q Sepharose™ XL, DEAE Sepharose™ FF, POROS® HQ50, Toyopearl® DEAE, Toyopearl® GigaCap Q-650M, Toyopearl® DEAE-650M, Capto™ Q, Capto™ DEAE, and tentacle anion exchange chromatography (e.g., Fractogel® TMAE HiCap (M)™, Fractogel® TMAE (S)™, or Fractoprep® TMAE™). In some embodiments, the anion exchange chromatography resin is a charged membrane adsorber (e.g., Mustang® Q, Mustang® E, Sartobind® and/or Chromasorb®). In some embodiments, the ion exchange chromatography resin is a charged monolithic support (e.g., CIM®-DISK). In particular embodiments, the affinity chromatography is MabSelect™ rProtein A affinity chromatography, the ion exchange chromatography is tentacle anion exchange chromatography, and the hydroxyapatite chromatography is Type I ceramic hydroxyapatite chromatography. In some embodiments, a method according to the invention involves no more than 3 chromatography steps. In some embodiments, a method according to the present invention further includes a step of stripping and/or regenerating one or more chromatography columns for reuse.

In some embodiments, the present invention can be used to purify a protein preparation containing more than 5% (e.g., more than 10%, 20%, 30%, 40%, 50%, 60%, 70%, or more) high molecular weight aggregates. In some embodiments, the present invention can be used to purify a protein preparation containing less than 70% (e.g., less than 60%, 50%, 40%, 30%, 20%, 15%, 10%, or 5%) high molecular weight aggregates. In some embodiments, the present invention can be used to purify a protein preparation containing 4-70% (e.g., 4-60%, 4-50%, 4-40%, 4-30%, 4-20%, 4-15%, 4-10%) high molecular weight aggregates.

In some embodiments, the present invention is used to purify a small modular immunopharmaceutical protein that binds specifically to CD20. In some embodiments, the present invention is used to purify a small modular immunopharmaceutical protein that comprises an amino acid sequence having at least 80% identity to any one of SEQ ID NOs:1-59 and 67-76.

In still another aspect, the present invention is used to purify a protein from a protein preparation containing more than 20% (e.g., more than 25%, 30%, 35%, 40%, 45%, 50%, 55%, 60%, 70%, or more) high molecular weight aggregates including a step of subjecting the protein preparation to hydroxyapatite chromatography under an operating condition such that the purified protein contains less than 4% (e.g., less than 3.5%, 3%, 2.5%, 2%, 1.5%, 1%, 0.8%, 0.6%, 0.5%, 0.4%, 0.2%, or 0.1%) aggregates. In certain embodiments, the protein preparation contains more than 60% high molecular weight aggregates.

In certain embodiments, the operating condition comprises eluting the protein from a hydroxyapatite chromatography column in a phosphate buffer. In some embodiments, the phosphate buffer is endotoxin-free. In some embodiments, the phosphate buffer is depyrogenated. In some embodiments, the phosphate buffer comprises sodium phosphate, potassium phosphate, and/or lithium phosphate. In some embodiments, the phosphate buffer comprises sodium phosphate at a concentration ranging from 1 mM to 50 mM. In some embodiments, the phosphate buffer further comprises sodium chloride at a concentration ranging from 100 mM to 2.5 M. In particular embodiments, the phosphate buffer comprises sodium phosphate at a concentration ranging from 2 mM to 32 mM and sodium chloride at a concentration ranging from 100 mM to 1.6 M. In some embodiments, the phosphate buffer has a pH ranging from 6.5 to 8.5.

In some embodiments, the protein to be purified contains a small modular immunopharmaceutical polypeptide.

The present invention further provides a small modular immunopharmaceutical protein purified using methods described herein. In addition, the present invention provides pharmaceutical compositions comprising a small modular immunopharmaceutical protein and a pharmaceutically acceptable carrier, wherein the small modular immunopharmaceutical protein comprises less than 4% (e.g., less than 3.5%, 3%, 2.5%, 2%, 1.5%, 1%, 0.8%, 0.6%, 0.5%, 0.4%, 0.2%, or 0.1%) aggregates.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only, not for limitation.

FIG. 1 depicts an exemplary structure of an anti-CD20 small modular immunopharmaceutical protein.

FIG. 2 illustrates exemplary configurations of SMIP™ molecules that may be in solution.

FIG. 3A-3C illustrate that various domain-swapping mechanisms may lead to the formation of high molecule weight aggregates of SMIP™ molecules, such as trimers, tetramers or multimers.

FIG. 4 depicts a schematic diagram illustrating an exemplary cell culture and harvest procedure.

FIG. 5 depicts exemplary daily titer measurements (μg/mL) of the production bioreactor of TRU-015 produced by two different CHO cell clones over a 12-14 day culture period. Peak titer values were obtained between days 12 and 14 of production bioreactor growth. Peak titer values ranged from 1500 to 3000 μg/mL.

FIG. 6 depicts an exemplary design of high throughput screening using batch binding mechanism.

FIG. 7 depicts an exemplary design of Protein A column operation and high throughput screening model.

FIG. 8 depicts exemplary Protein A high-throughput screen results.

FIG. 9. (A) Summary of exemplary variables tested in high-throughput screens for ceramic hydroxyapatite chromatography. (B) Exemplary contour plot demonstrating percent HMW compounds when varying concentrations of phosphate and NaCl were used during cHA purification. (C) Exemplary HMW vs. Log Kp plot demonstrating that at a Kp of approximately 10 (or log Kp of 1), most of the HMW compounds have been removed from the sample.

FIG. 10 depicts an exemplary alternative screening using a cHA column and a NaCl gradient elution for the development of the cHA chromatography step.

FIG. 11 depicts an exemplary typical cHA chromatogram.

FIG. 12 depicts an exemplary TRU-015 purification process.

FIG. 13 depicts an exemplary comparison of reduction of amount of HMW aggregates by MabSelect Protein A affinity chromatography with that by CEX.

FIG. 14 depicts exemplary results illustrating protein product binding capacities of CEX resins.

FIG. 15 depicts exemplary results illustrating CEX peaks using 25 vs. 75 mg/mLr loading challenge.

FIG. 16 depicts an exemplary result illustrating effective removal of HMW using an AEX column. The collected pool was 88% pure with >95% yield of the “monomeric” SMIP™ protein.

### DETAILED DESCRIPTION OF THE INVENTION

The present invention, among other things, provides methods of purifying or recovering proteins, in particular, small modular immunopharmaceutical proteins, from protein preparations containing HMW aggregates and other impurities based on hydroxyapatite chromatography. In some embodiments, the hydroxyapatite chromatography is used in combination with affinity chromatography and/or ion exchange chromatography. In some embodiments, inventive methods of the present invention further include one or more filtration steps to further remove viral contaminants, to concentrate proteins, and/or buffer exchange. In some embodiments, the methods of the invention have no more than three chromatography steps (e.g., two chromatography steps, or three chromatography steps). In some embodiments, the methods of the invention have no more than 3 filtration steps (e.g., two filtration steps, three filtration steps).

As described in the Examples section, the present inventors have discovered suitable operating conditions for hydroxyapatite chromatography, affinity chromatography and/or ion exchange chromatography that allow effective removal of HMW aggregates and other impurities (e.g., DNA, host cell protein, viruses, and other contaminants) from protein preparations. In some embodiments, the percentage of HMW aggregates can be reduced from more than 20% (e.g., 25%, 30%, 35%, 40%, 45%, 50%, 55%, 60%, 70% or more) in a starting preparation to less than 4% (e.g., less than 3.5%, 3.0%, 2.5%, 2.0%, 1.5%, 1.0%, 0.8%, 0.6%, 0.4%, 0.2%, 0.1%) in the purified protein product. In some embodiments, the HMW aggregates in a starting preparation can be reduced by at least about 5 fold, or at least about 10 fold, or at least about 20 fold, or at least about 30 fold, or at least about 40 fold, or at least about 50 fold, or at least about 60 fold, or at least about 70 fold, or at least about 80 fold, or at least about 90 fold, or at least about 100 fold. Additionally or alternatively, the percentage of other contamination (e.g., HCP) in the purified protein is not more than about 10,000 ppm, or not more than about 5000 ppm, or not more than about 2500 ppm, or not more than about 400 ppm, or not more than about 360 ppm, or not more than about 320 ppm, or not more than about 280 ppm, or not more than about 240 ppm, or not more than about 200 ppm, or not more than about 160 ppm, or not more than about 140 ppm, or not more than about 120 ppm, or not more than about 100 ppm, or not more than about 80 ppm, or not more than about 60 ppm, or not more than about 40 ppm, or not more than about 30 ppm, or not more than about 20 ppm, or not more than about 10 ppm.

In some embodiments, inventive methods according to the invention provide at least 50% recovery of the protein of interest (e.g., at least 55%, 60%, 65%, 70%, 75%, 80%, 85%, 90%, or 95%). In some embodiments, the methods of the invention provide at least 20% product yield (e.g., at least 22%, 24%, 26%, 28%, 30%, 32%, 34%, 36%, 38%, 40%, 42%, 44%, 46%, 48%, or 50%).

Various aspects of the invention are described in detail in the following sections. The use of sections is not meant to limit the invention. Each section can apply to any aspect of the invention. In this application, the use of “or” means “and/or” unless stated otherwise.

### DEFINITIONS

In order for the present invention to be more readily understood, certain terms are first defined. Additional definitions for the following terms and other terms are set forth throughout the specification.

Absorbent: An absorbent is at least one molecule affixed to a solid support or at least one molecule that is, itself, a solid, which is used to perform chromatography.

Affinity chromatography: Affinity chromatography is chromatography that utilizes the specific, reversible interactions between biomolecules, for example, the ability of Protein A to bind to an Fc portion of an IgG antibody, rather than the general properties of a molecule, such as isoelectric point, hydrophobicity, or size, to effect chromatographic separation. In practice, affinity chromatography involves using an absorbent, such as Protein A affixed to a solid support, to chromatographically separate molecules that bind more or less tightly to the absorbent. See Ostrove (1990) in Guide to Protein Purification, Methods in Enzymology 182: 357-379, which is incorporated herein in its entirety.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bind-elute mode: The term “bind-elute mode” (also referred to as “binding mode”) refers to a product preparation separation technique in which at least one product contained in the preparation binds to a chromatographic resin or medium. The bound product in this mode is eluted during the elution phase.

Chromatography: Chromatography is the separation of chemically different molecules in a mixture from one another by percolation of the mixture through an absorbent, which absorbs or retains different molecules more or less strongly. Molecules that are least strongly absorbed to or retained by the absorbent are released from the absorbent under conditions where those more strongly absorbed or retained are not.

Constant immunoglobulin domain: A constant antibody immunoglobulin domain is an immunoglobulin domain that is identical to or substantially similar to a C_{L}, C_{H1}, C_{H2}, C_{H3}, or C_{H4} domain of human or animal origin. See e.g. Charles A Hasemann and J. Donald Capra, Immunoglobulins: Structure and Function, in William E. Paul, ed., Fundamental Immunology, Second Edition, 209, 210-218 (1989), which is incorporated by reference herein in its entirety. A C_{H2} or C_{H3} domain, or an immunoglobulin domain substantially similar to C_{H2} or C_{H3} domain, is also referred to as the F_{C} portion of an antibody.

Contaminant or Impurity: A contaminant or an impurity refers to any foreign or objectionable molecule, including a biological macromolecule such as a DNA, an RNA, or a protein, other than the protein of interest being purified that is also present in a sample of the protein of interest being purified. Impurities include, for example, protein variants, such as aggregated proteins, high molecular weight species, low molecular weight species and fragments, and deamidated species; other proteins from host cells that secrete the protein being purified (host cell proteins); proteins that are part of an absorbent used for affinity chromatography that may leach into a sample during prior purification steps, such as Protein A; endotoxins; and viruses.

Flow-through mode: The term “flow-through mode” generally refers to a product preparation separation technique in which at least one product contained in the preparation is intended to flow through a chromatographic resin or medium, while at least one potential contaminant or impurity binds to the chromatographic resin or medium. In some embodiments, a flow-through mode is weak partitioning chromatography (WPC), in which the product can bind weakly to the resin, while at least one potential contaminant or impurity binds more preferentially to the chromatographic resin or medium. Typically, WPC operates at a higher partition coefficient than in traditional flow-through mode, but at a partition coefficient lower than a bind-and-elute mode. In weak partitioning, high recoveries can be achieved with larger load challenges and short washes applied following the load phase.

Host cell proteins: Host cell proteins are proteins encoded by the naturally-occurring genome of a host cell into which DNA encoding a protein that is to be purified is introduced. Host cell proteins may be contaminants of the protein to be purified, the levels of which may be reduced by purification. Host cell proteins can be assayed for by any appropriate method including gel electrophoresis and staining and/or ELISA assay, among others.

Hydroxyapatite chromatography: Hydroxyapatite chromatography is chromatography using ceramic hydroxyapatite as an absorbent. See e.g. Marina J. Gorbunoff (1990), Protein Chromatography on Hydroxyapatite Columns, in Guide to Protein Purification, Murray P. Deutscher, ed., Methods in Enzymology 182: 329-339, which is incorporated herein in its entirety.

Load: The term “load” refers to any load material containing the product, either derived from clarified cell culture or fermentation conditioned medium, or a partially purified intermediate derived from a chromatography step. The term “load fluid” refers to a liquid containing the load material, for passing through a medium under the operating conditions of the invention.

Load challenge (LC): The term “load challenge” refers to the total mass of product loaded onto the column in the load cycle of a chromatography step or applied to the resin in batch binding, measured in units of mass of product per unit volume of resin.

Protein A: Protein A is a protein originally discovered in the cell wall of Stapphylococcus that binds to an F_{C} portion or a variable domain of an antibody. In some embodiments, Protein A binds to a domain from VH₃ family (e.g., a VH₃ domain of IgG antibody). For purposes of the invention, “Protein A” is any protein identical or substantially similar to Stapphylococcal Protein A, including commercially available and/or recombinant forms of Protein A. For purposes of the invention, the biological activity of Protein A for the purpose of determining substantial similarity is the capacity to bind to an F_{C} portion or a variable domain (e.g., VH₃) of IgG antibody.

Protein G: Protein G is a protein originally discovered in the cell wall of Streptococcus that binds to an F_{C} portion or a variable domain of an antibody (e.g., IgG). In some embodiments, Protein G binds to a domain from VH₃ family (e.g., a VH₃ domain of IgG antibody). For purposes of the invention, “Protein G” is any protein identical or substantially similar to Streptococcal Protein G, including commercially available and/or recombinant forms of Protein G. For purposes of the invention, the biological activity of Protein G for the purpose of determining substantial similarity is the capacity to bind to an F_{C} portion or a variable domain (e.g., VH₃) of IgG antibody.

Protein LG: Protein LG is a recombinant fusion protein that binds to IgG antibodies comprising portions of both Protein G (see definition above) and Protein L. Protein L was originally isolated from the cell wall of Peptostreptococcus. Protein LG comprises IgG binding domains from both Protein L and G. Vola et al. (1994) Cell. Biophys. 24-25: 27-36, which is incorporated herein in its entirety. For purposes of the invention, “Protein LG” is any protein identical or substantially similar to Protein LG, including commercially available and/or recombinant forms of Protein LG. For purposes of the invention, the biological activity of Protein LG for the purpose of determining substantial similarity is the capacity to bind to an IgG antibody.

Purify: To purify a protein means to reduce the amounts of foreign or objectionable elements, especially biological macromolecules such as proteins or DNA, that may be present in a sample of the protein. The presence of foreign proteins may be assayed by any appropriate method including gel electrophoresis and staining and/or ELISA assay. The presence of DNA may be assayed by any appropriate method including gel electrophoresis and staining and/or assays employing polymerase chain reaction.

Variable antibody immunoglobulin domain: A variable antibody immunoglobulin domain is an immunoglobulin domain that is identical or substantially similar to a V_{L} or a V_{H} domain of human or animal origin. For purposes of the invention, the biological activity of a variable antibody immunoglobulin domain for the purpose of determining substantial similarity is antigen binding. In some embodiments, a variable antibody immunoglobulin domain is a VH₃ domain. A VH₃ domain, as used herein refers to VH₃ itself, or any domain having homology to the VH₃ domain.

### Small Modular Immunopharmaceutical Proteins

As used herein, a small modular immunopharmaceuticals (SMIP™) protein refers to a protein that contains one or more of the following fused domains: a binding domain, an immunoglobulin hinge region or a domain derived therefrom, and an effector domain, which can be an immunoglobulin heavy chain C_{H2} constant region or a domain derived therefrom, and an immunoglobulin heavy chain C_{H3} constant region or a domain derived therefrom. SMIP™ protein therapeutics are preferably mono-specific (i.e., they recognize and attach to a single antigen target to initiate biological activity). The present invention also relates to multi-specific and/or multi-valent molecules such as SCORPION™ therapeutics, which incorporate a SMIP™ protein and also have an additional binding domain located C-terminally to the SMIP™ protein portion of the molecule. Preferably, the binding domains of SCORPION™ therapeutics each bind to a different target. The domains of small modular immunopharmaceuticals suitable for the present invention are, or are derived from, polypeptides that are the products of human gene sequences, any other natural or artificial sources, including genetically engineered and/or mutated polypeptides. Small modular immunopharmaceuticals are also known as binding domain-immunoglobulin fusion proteins.

In some embodiments, a hinge region suitable for a small modular immunopharmaceutical is derived from an immunoglobulin such as IgG1, IgA, IgE, or the like. For example, a hinge region can be a mutant IgG1 hinge region polypeptide having either zero, one or two cysteine residues.

A binding domain suitable for a small modular immunopharmaceutical protein may be any polypeptide that possesses the ability to specifically recognize and bind to a cognate biological molecule, such as an antigen, a receptor (e.g., CD20), or complex of more than one molecule or assembly or aggregate.

Binding domains may include at least one immunoglobulin variable region polypeptide, such as all or a portion or fragment of a heavy chain or a light chain V-region, provided it is capable of specifically binding an antigen or other desired target structure of interest. In other embodiments, binding domains may include a single chain immunoglobulin-derived Fv product, which may include all or a portion of at least one immunoglobulin light chain V-region and all or a portion of at least one immunoglobulin heavy chain V-region, and which further comprises a linker fused to the V-regions.

The present invention can be applied to various small modular immunopharmaceuticals. Exemplary small modular immunopharmaceuticals may target receptors or other proteins, such as, CD3, CD4, CD8, CD19, CD20 and CD34; members of the HER receptor family such as the EGF receptor, HER2, HER3 or HER4 receptor; cell adhesion molecules such as LFA-1, Mol, p150,95, VLA-4, ICAM-1, VCAM, growth factors such as VEGF; IgE; blood group antigens; flk2/flt3 receptor; obesity (OB) receptor; protein C; EGFR, RAGE, P40, Dkk1, NOTCH1, IL-13, IL-21, IL-4, and IL-22, etc.

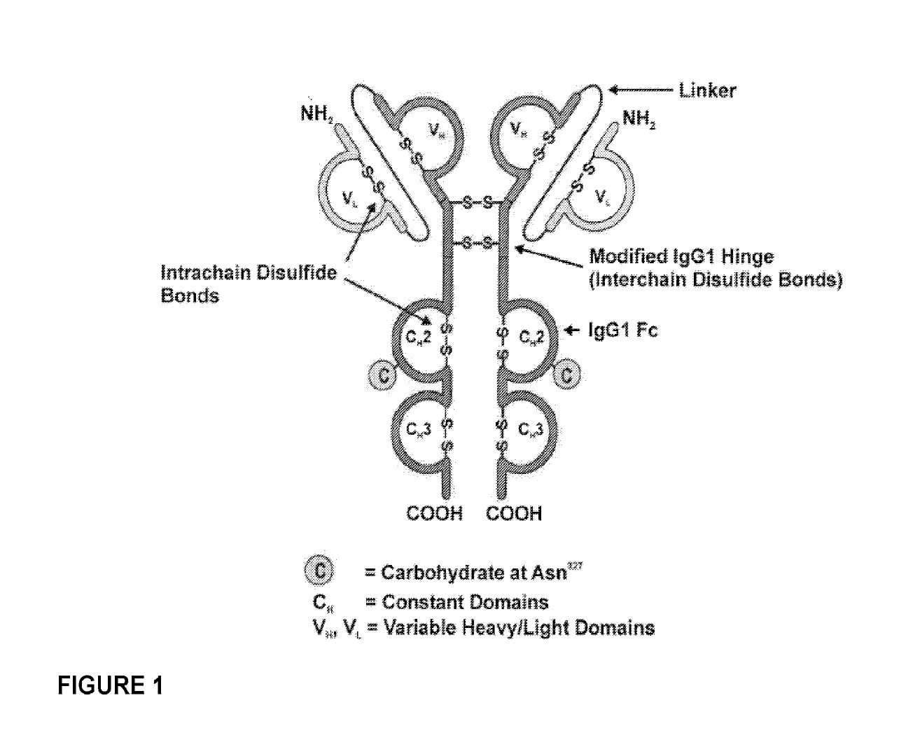

In some embodiments, the present invention is utilized to purify small modular immunopharmaceuticals that specifically recognize CD20. An exemplary small modular immunopharmaceutical protein that specifically binds CD20 is shown in FIG. 1. As shown in FIG. 1, an anti-CD20 SMIP™ protein is typically a recombinant homodimeric fusion protein composed of three distinct domains: (1) a chimeric (murine/human) CD20 binding domain including the variable heavy (VH) and light (VL) chain fragments connected by an amino acid linker (e.g., a 15-amino acid linker); (2) a modified human immunoglobulin (e.g., IgG1) hinge domain and, (3) an IgG effector domain such as the CH2 and CH3 domains of human IgG1.

Typically, an SMIP™ protein may exist in two distinctly associated homodimeric forms, the major form, which is the predicted interchain disulfide linked covalent homodimer (CD), and a homodimeric form that does not possess interchain disulfide bonds (dissociable dimer, DD). The dissociable dimer is generally fully active. Typically, a dimer has a theoretical molecular weight of approximately 106,000 daltons. SMIP™ proteins can also form multivalent complexes.

Typically, SMIP™ proteins are present as glycoproteins. For example, as shown in FIG. 1, an anti-CD20 SMIP™ protein may be modified with oligosaccharides at the N-linked glycosylation consensus sequence (e.g., ³²⁷NST) in the CH2 domain of each protein chain (see FIG. 1). SMIP™ proteins may also contain a core-fucosylated asialo-agalacto-biantennary N-linked oligosaccharide (G0F); COOH-terminal G1y⁴⁷⁶, and NH2-terminal pyroglutamate on each chain (G0F/G0F). Two minor glycoforms, G1F/G0F and G1F/G1F, and other expected trace-level N-linked glycoforms may also present. Additionally, low levels of a Core 1 O-glycan modification is also observed in the hinge region of SMIP™ proteins.

In some embodiments, the isoelectric point (pI or IEP) of SMIP™ proteins ranges from approximately 7.0 to 9.0 (e.g., 7.2, 7.4, 7.6, 7.8, 8.0, 8.2, 8.4, 8.6, 8.8).

The present invention can be used to purify SMIP™ proteins in various forms as discussed herein (e.g., monomeric polypeptide, homodimer, dissociable dimer or multivalent complexes). The present invention can be used to purify various modified SMIP™ proteins, such as humanized SMIP™, or chimeric SMIP™ proteins. As used herein, the term “humanized SMIP™ proteins” refers to SMIP™ proteins that include at least one humanized immunoglobulin region (e.g., humanized immunoglobulin variable or constant region). In some embodiments, a humanized SMIP™ protein comprises a humanized variable region that includes a variable framework region derived substantially from a human immunoglobulin (e.g., a fully human FR1, FR2, FR3, and/or FR4), while maintaining target-specific one or more complementarity determining regions (CDRs) (e.g., at least one CDR, two CDRs, or three CDRs). In some embodiments, a humanized SMIP™ protein comprises one or more human or humanized constant regions (e.g., human immunoglobulin C_{H2} and/or C_{H3} domains). The term “substantially from a human immunoglobulin or antibody” or “substantially human” means that, when aligned to a human immunoglobulin or antibody amino sequence for comparison purposes, the region shares at least 80-90%, preferably 90-95%, more preferably 95-99% identity (i.e., local sequence identity) with the human framework or constant region sequence, allowing, for example, for conservative substitutions, consensus sequence substitutions, germline substitutions, backmutations, and the like. As used herein, the term “chimeric SMIP™ proteins” refers to SMIP™ proteins whose variable regions derive from a first species and whose constant regions derive from a second species. Chimeric SMIP™ proteins can be constructed, for example by genetic engineering, from immunoglobulin gene segments belonging to different species. Humanized and chimeric SMIP™ proteins are further described in International Application Publication No. WO 2008/156713, which is incorporated by reference herein.

The present invention can also be used to purify SMIP™ proteins with modified glycosylation patterns and/or mutations to the hinge, C_{H2} and/or C_{H3} domains that alter the effector functions. In some embodiments, SMIP™ proteins may contain mutations on adjacent or close sites in the hinge link region that affect affinity for receptor binding. In addition, the invention can be used to purify fusion proteins including a small modular immunopharmaceutical polypeptide or a portion thereof.

In some embodiments, the present invention can be used to purify SMIP™ proteins that include an amino acid sequence of any one of SEQ ID NOs:1-76 (see the Exemplary SMIP™ Sequences section), or a variant thereof. In some embodiments, the present invention can be used to purify SMIP™ proteins that contain a variable domain having an amino acid sequence of any one of SEQ ID NOs:1-59 or a variant thereof. In some embodiments, the present invention can be used to purify SMIP™ proteins that contain a variable domain having an amino acid sequence of any one of SEQ ID NOs:1-59 or a variant thereof, a hinge region having an amino acid sequence of any one of SEQ ID NOs: 60-64 or a variant thereof, and/or an immunoglobulin constant region having an amino acid sequence of SEQ ID NO: 65 or 66 or a variant thereof. In some embodiments, the present invention can be used to purify SMIP™ proteins that have an amino acid sequence of any one of SEQ ID NOs: 67-76, or a variant thereof.

As used herein, variants of a parent sequence include, but are not limited to, amino acid sequences that are at least 70%, 75%, 80%, 85%, 90%, 95%, 96%, 97%, 98%, 99%, identical to the parent sequence. The percent identity of two amino acid sequences can be determined by visual inspection and mathematical calculation, or more preferably, the comparison is done by comparing sequence information using a computer program such as the Genetics Computer Group (GCG; Madison, Wis.) Wisconsin package version 10.0 program, “GAP” (Devereux et al., 1984, Nucl. Acids Res. 12: 387) or other comparable computer programs. The preferred default parameters for the ‘GAP’ program includes: (1) the weighted amino acid comparison matrix of Gribskov and Burgess ((1986), Nucl. Acids Res. 14: 6745), as described by Schwartz and Dayhoff, eds., Atlas of Polypeptide Sequence and Structure, National Biomedical Research Foundation, pp. 353-358 (1979), or other comparable comparison matrices; (2) a penalty of 30 for each gap and an additional penalty of 1 for each symbol in each gap for amino acid sequences; (3) no penalty for end gaps; and (4) no maximum penalty for long gaps. Other programs used by those skilled in the art of sequence comparison can also be used.

Additional small modular immunopharmaceuticals are further described in, e.g., US Patent Publications 20030133939, 20030118592, 20040058445, 20050136049, 20050175614, 20050180970, 20050186216, 20050202012, 20050202023, 20050202028, 20050202534, 20050238646, and 20080213273; International Patent Publications WO 02/056910, WO 2005/037989, and WO 2005/017148, which are all incorporated by reference herein.

### Protein Aggregation

Without wishing to be bound by any theory, it is contemplated that domain swapping may be a protein aggregation mechanism. Domain swapping occurs when a distinctly structured subsection of a protein (domain) physically exchanges with that of another monomer to create a dimer or higher oligomers. In domain-swapped proteins, each domain maintains native-like global structure that is comparable to its structure in the un-swapped monomer. When a folded protein, containing multiple domains, is stressed by low pH, elevated temperature or shear force a partially folded or “open” conformation (characterized by dissociated, but folded domains) can be induced. Some “open” molecules refold to their native structure, by simple re-association of the folded domains. In some cases (usually favored by higher protein concentrations) the domain re-association occurs between two neighboring molecules, resulting in a domain-swapped dimer. Such inter-molecular swapping may propagate, leading to larger aggregates. Typically, domain-swapped proteins are non-covalently (but stably) associated molecules, having native-like domain folding and inter-domain contacts. In such cases, multimeric proteins are held together by the very same domain-domain interfaces that would normally exist intra-molecularly.

Prior to the purification process, SMIP™ proteins contain a significant amount (e.g., 20-60%) of HMW protein (aggregate). The excessive HMW content may be due to the molecular structure of SMIPs™. As shown in FIG. 1, a typical SMIP™ dimer molecule contains 2 identical single-chain-Fv regions, including V_{H} and V_{L} domains connected by a flexible linker (e.g., GGGSGGGGSGGS (SEQ ID NO: 77)), which are fused to a human IgG1 Fc domain (FIG. 1). Without wishing to be bound by any theory, SMIP™ molecules may be more susceptible to unfolding (open conformation of the Fv) and subsequent domain swapping resulting in protein aggregation because of the flexible linker in each subunit.

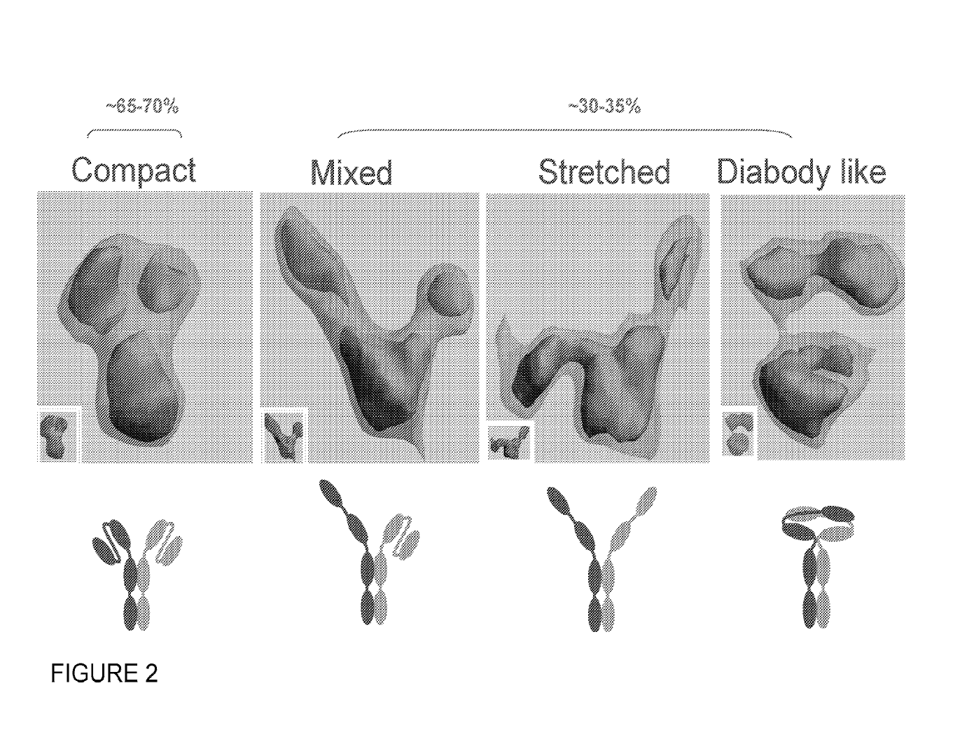

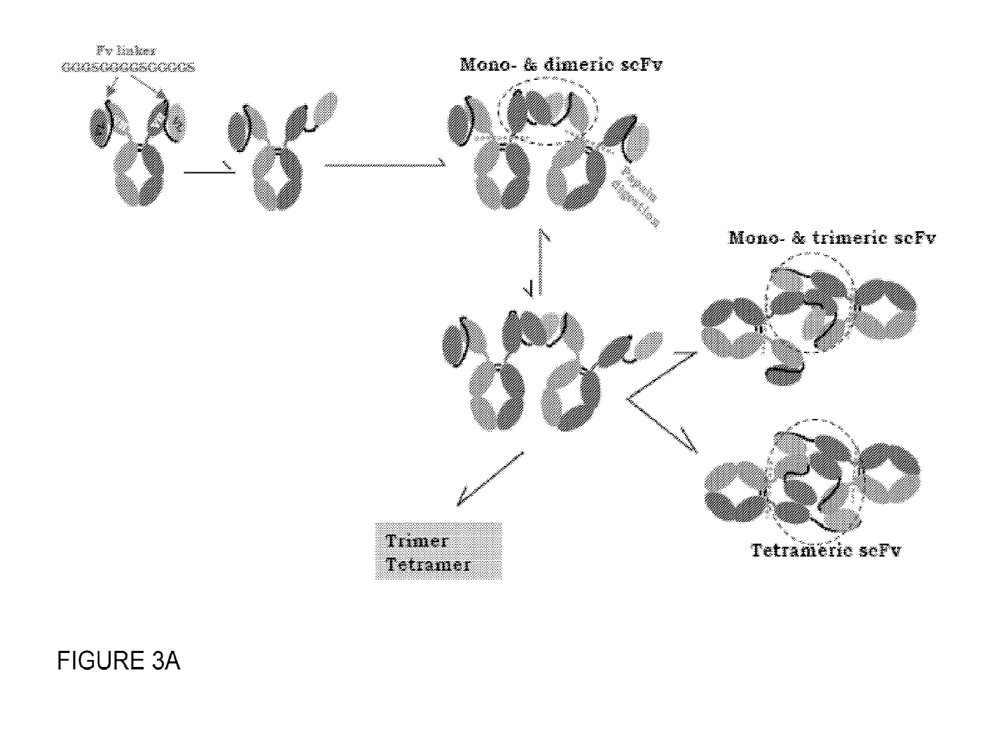

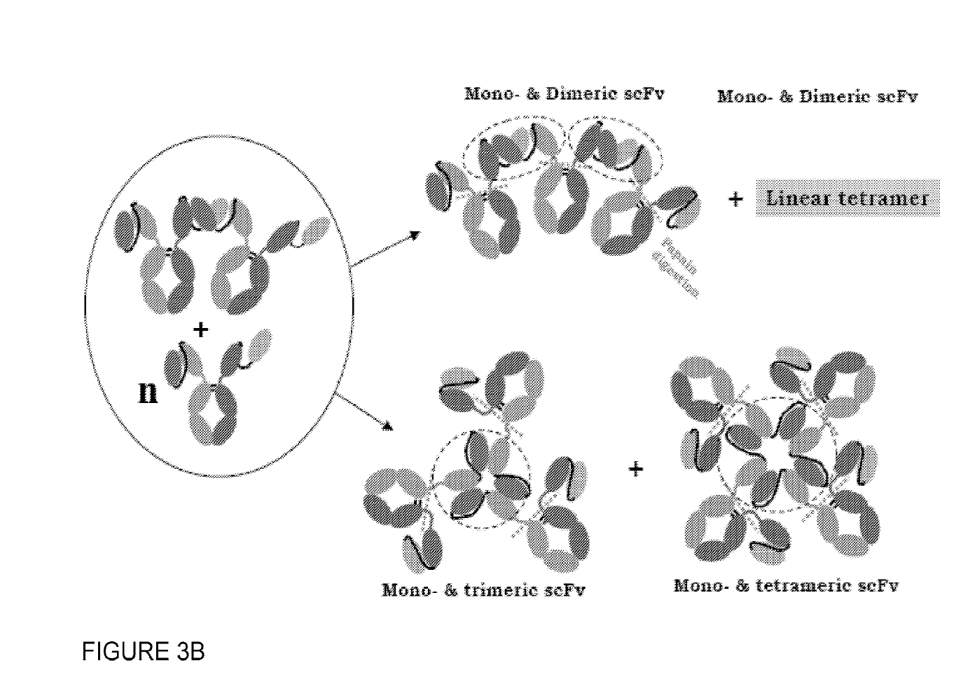

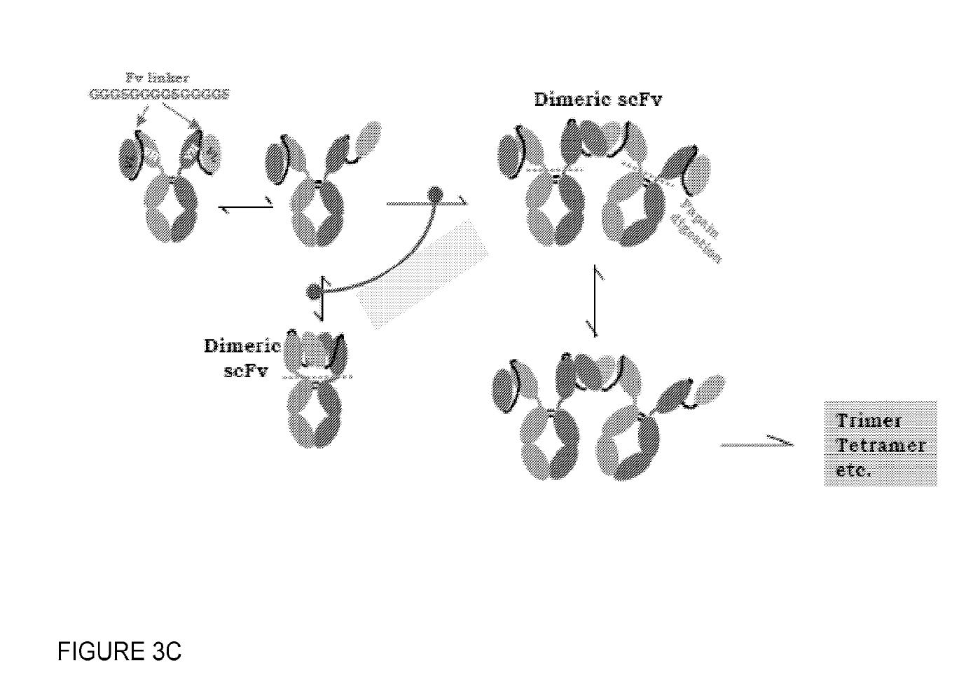

According to studies using cryo-electron microscope, SMIP™ molecules may exist in, e.g., compact, mixed, stretched or diabody-like configurations in solution (FIG. 2). Without wishing to be bound by any theory, it is contemplated that some SMIP™ molecules with stretched or open chains may attempt to refold to their native structure, by simple re-association of the folded domains. As shown in FIG. 3A, the domain re-association may occur between two neighboring SMIP™ molecules, resulting in a domain-swapped dimer. Such inter-molecular swapping may propagate, leading to larger aggregates, such as trimers, tetramers or multimers (see, FIGS. 3B and 3C).

### Protein Preparations

Protein preparations used with methods described herein can be obtained from any in vivo or in vitro protein expression systems. Exemplary sources for protein preparation suitable for the invention include, but are not limited to, conditioned culture medium derived from culturing a recombinant cell line that expresses a protein of interest, or from a cell extract of, e.g., product-producing cells, bacteria, fungal cells, insect cells, transgenic plants or plant cells, transgenic animals or animal cells, or serum of animals, ascites fluid, hybridoma or myeloma supernatants. Suitable bacterial cells include, but are not limited to, Escherichia coli cells. Examples of suitable E. coli strains include: HB101, DH5α, GM2929, JM109, KW251, NM538, NM539, and any E. coli strain that fails to cleave foreign DNA. Suitable fungal host cells that can be used include, but are not limited to, Saccharomyces cerevisiae, Pichia pastoris and Aspergillus cells. Suitable insect cells include, but are not limited to, S2 Schneider cells, D. Mel-2 cells, SF9, SF21, High-5™, Mimic™-SF9, MG1 and KC1 cells. Suitable exemplary recombinant cell lines include, but are not limited to, BALB/c mouse myeloma line, human retinoblasts (PER.C6), monkey kidney cells, human embryonic kidney line (293), baby hamster kidney cells (BHK), Chinese hamster ovary cells (CHO), mouse sertoli cells, African green monkey kidney cells (VERO-76), human cervical carcinoma cells (HeLa), canine kidney cells, buffalo rat liver cells, human lung cells, human liver cells, mouse mammary tumor cells, TRI cells, MRC 5 cells, FS4 cells, and human hepatoma line (Hep G2).

Proteins of interest can be expressed using various vectors (e.g., viral vectors) known in the art and cells can be cultured under various conditions known in the art (e.g., fed-batch). Various methods of genetically engineering cells to produce proteins are well known in the art. See e.g. Ausabel et al., eds. (1990), Current Protocols in Molecular Biology (Wiley, New York).

Cells expressing SMIP™ proteins may be cultured in various cell culture media known in the art. Exemplary cell culture media may be based on DMEM, DMEM/F12, Ham's F-10, Ham's F-12, F-12K, Medium 199, MEM, RPMI 1640, Ames', BGJb, Click's, CMRL-1066, Fischers, GMEM, IMDM, L-15, McCoy's 5A Modified, NCTC, Swik's S-77, Waymouth, William's Medium E. Suitable cell culture medium may be serum free. In some embodiments, suitable cell culture medium may include serum/culture medium additives including, but not limited to, fetal bovine serum, newborn bovine serum, calf bovine serum, and adult bovine serum, chicken, goat, horse, porcine, rabbit, sheep, human serum, serum replacement or bovine embryonic fluid. Suitable cell culture medium may further include supplements and/or antibiotics including, but not limited to, L-Glutamine Solution, L-Albany-L-Glutamine Solution, Non-essential Amino Acid Solution, Penicillin, Streptomycin.

The present invention can be utilized to purify crude protein preparations. For example, the present invention can be used to purify proteins directly from conditioned culture medium containing proteins secreted from cultured cells. Conditioned culture medium can be obtained from small scale cultures (e.g., shake flasks, wavebags), or from seed bioreactors or production bioreactors (e.g., 250 L, 600 L, 2500 L, or 6000 L bioreactors). In some embodiments, the present invention can be utilized to purify proteins expressed intracellularly from crude cell lysates prepared from protein-containing cells. In some embodiments, the present invention can be used to purify proteins from serum, milk or other fluid containing protein of interest. In some embodiments, the present invention can be used to purify proteins from partially purified preparations such as eluates or flow-through from chromatography columns, or intermediate protein preparations from storage or formulation processes.

In some embodiments, the present invention can be used to purify proteins that are expressed in inclusion bodies (e.g., bacterial, viral, plant cell or any other types of inclusion bodies). Proteins expressed in inclusion bodies typically form aggregates, which pose challenges for purification. The present invention therefore can be particularly useful for purifying proteins expressed in inclusion bodies. Purification of proteins from inclusion bodies usually requires first extracting inclusion bodies from bacteria or other type of cells followed by solubilizing the purified inclusion bodies. Various methods of inclusion body extraction and solubilization are well known in the art and can be used in the present invention. For example, strong denaturing agents (e.g., urea and guanidine hydrochloride), altered pH and/or temperature, physical disruptions (e.g., sonication, etc.), among others can be used to extract and/or solubilize inclusion bodies. Inclusion body extraction and/or solubilization process may lead to mis-folded proteins. In some embodiments, inclusion body extracts can be directly loaded to chromatography columns according to the present invention. In some embodiments, the proteins extracted from inclusion bodies are first subjected to a refolding process prior to chromatography steps described herein. In some embodiments, a refolding process may include dialysis or dilution of the proteins into a folding buffer. Various folding buffers are well known in the art and can be used in the present invention.

In some embodiments, the present invention can be used to purify proteins from preparations that contain various impurities including, but not limited to, undesirable protein variants, such as aggregated proteins, e.g., high molecular weight species, low molecular weight species and fragments, and deamidated species; other proteins from host cells that secrete the protein being purified; host cell DNA; components from the cell culture medium, molecules that are part of an absorbent used for affinity chromatography that leach into a sample during prior purification steps, for example, Protein A and Protein G; an endotoxin; a nucleic acid; a virus, or a fragment of any of the forgoing.

The present invention is particularly useful to remove HMW aggregates. In some embodiments, starting protein preparations may contain at least 4%, 5%, 10%, 15%, 20%, 25%, 30%, 35%, 40%, 45%, 50%, 55%, 60%, 65%, 70%, 75%, 80%, 85%, 90%, 95% HMW aggregates. In some embodiments, starting protein preparations may contain less than 95%, 90%, 85%, 80%, 75%, 70%, 65%, 60%, 55%, 50%, 45%, 40%, 35%, 30%, 25%, 20%, 15%, 10%, 5% HMW aggregates. In some embodiments, starting preparations may contain HMW aggregates in a range of the above percentage combination (for example, about 4-95%, 5-70%, 10-60%, 4-30%, 4-25%, 4-20%, 4-15%, 4-10%, and any combinations of the above identified percentages). As used herein, the term “high molecular weight (HMW) aggregates” refers to an association of at least two protein monomers. For the purposes of this invention, a monomer refers to the single unit of any biologically active form of the protein of interest. For example, a monomer of a small modular immunopharmaceutical protein can be a monomeric polypeptide, or a homodimer, or a dissociable dimer, or a unit of multivalent complex of SMIP™ protein. The association may be covalent, non-covalent, disulfide, non-reducible crosslinking, or by other mechanism.

In some embodiments, appropriate protein preparations can be obtained by harvest processing. As discussed in the Examples, conditioned medium can be harvested from production bioreactors through centrifugation (e.g., by disc stack centrifugation (DSC)). A centrifugation step may separate cells and cell debris from conditioned medium containing secreted proteins (e.g., SMIPs™). In some embodiments, after DSC, the contents can be applied to a pad filtration apparatus, and then filters into a filtrate vessel or bag. In some embodiments, a Hepes/EDTA buffer solution can be added to the filtrated concentrate pool to reduce the generation of acidic species during the in-process hold period between the DSC and the affinity chromatography step. In addition, protease inhibitors such as EDTA or imidazole may be added to inhibit metalloprotease activity, certain serine protease or other protease activities. In some embodiments, a suitable protease inhibitor may be added to a protein preparation in an amount from about 0.001 μM to about 100 mM. The protease inhibitor(s) may be added to the protein preparations before and/or during protein A affinity chromatography. Adding protease inhibitors (e.g., EDTA) may also reduce protein A leaching. Other conditions such as temperature and pH may also be adjusted to reduce protein A leaching. Methods and conditions for reducing protein A leaching are described in details in US Publication No. 20050038231, which is incorporated by reference herein.

### Methods of Purification

Purification processes according to the invention involve one or more chromatography steps (e.g., affinity chromatography, hydroxyapatite chromatography, or ion exchange chromatography). In some embodiments, the purification methods of the invention involve a step of hydroxyapatite chromatography. In some embodiments, the purification methods of the invention involve a step of hydroxyapatite chromatography in combination of affinity chromatography and/or ion exchange chromatography. In some embodiments, the methods of the invention further include membrane filtration steps (e.g., ultrafiltration, diafiltration, and/or final filtration). Exemplary purification processes are described in details in the Examples section.

### Affinity Chromatography

The primary objectives of the affinity chromatography step include product capture from starting preparations (e.g., cell-free conditioned medium, cell lysates, inclusion body extracts, among others) and separation of protein of interest from process-derived impurities (e.g., host cell DNA and host cell proteins, medium components, and adventitious agents).

Thus, affinity chromatography suitable for the invention involves using an absorbent that can bind to a SMIP™ protein. For example, a suitable absorbent can be a protein that binds to a constant antibody immunoglobulin domain. Suitable absorbents can be Protein G, Protein LG, or Protein A. In some embodiments, a suitable absorbent is a protein that binds to a variable antibody immunoglobulin domain (e.g., a VH₃ domain or a domain homologous to a VH₃ domain). Absorbents can be affixed to any suitable solid support including: agarose, sepharose, silica, collodion charcoal, sand, and any other suitable material. Such materials are well known in the art. Any suitable method can be used to affix an absorbent to the solid support. Methods for affixing proteins to suitable solid supports are well known in the art. See e.g. Ostrove (1990), in Guide to Protein Purification, Methods in Enzymology, 182: 357-371.

In some embodiments, a suitable affinity chromatography step may use a Protein A chromatography column or a Protein G chromatography column. A Protein A chromatography column can be, for example, PROSEP-A™ (Millipore, U.K.), Protein A Sepharose FAST FLOW™ (GE Healthcare, Piscataway, N.J.), TOYOPEARL™ 650M Protein A (TosoHass Co., Philadelphia, Pa.), or MabSelect™ Protein A column (GE Healthcare, Piscataway, N.J.).

Before applying protein preparations to affinity chromatography columns, it may be desirable to adjust parameters such as pH, ionic strength, and temperature and in some instances the addition of substances of different kinds. Thus, it is an optional step to perform an equilibration of an affinity chromatography column by washing it with a solution (e.g., a buffer for adjusting pH, ionic strength, etc., or for the introduction of a detergent) bringing suitable characteristics for binding and purification of the protein product.

In some embodiments, the Protein A column may be equilibrated using a solution containing a salt, e.g., about 100 mM to about 150 mM sodium phosphate, about 100 mM to about 150 mM sodium acetate, and about 100 mM to about 150 mM NaCl. The pH of the equilibration buffer may range from about 6.0 to about 8.0. In one embodiment, the pH of the equilibration buffer is about 7.5. The equilibration buffer may also contain about 10 mM to about 50 mM Tris. In another embodiment, the buffer may contain about 20 mM Tris.

After a protein preparation is loaded, the bound column may be washed using a wash solution. Suitable wash solutions may contain salt (e.g., Hepes, sodium chloride, calcium chloride, magnesium chloride), arginine, histidine, Tris and/or other components. In some embodiments, a wash solution suitable for the invention may contain arginine or an arginine derivative. Suitable arginine derivative can be, but is not limited to, acetyl arginine, agmatine, arginic acid, N-alpha-butyroyl-L-arginine, or N-alpha-pyvaloyl arginine. A suitable concentration of arginine or arginine derivative in the wash solution is between about 0.1 M and about 2.0 M (e.g., 0.1 M, 0.4 M, 0.5 M, 1.0 M, 1.5 M, or 2.0 M), or between about 0.5 M and about 1.0 M (e.g., 0.5 M, 0.6 M, 0.7 M, 0.8 M, 0.9 M, or 1.0 M). The use of arginine or arginine derivative in affinity chromatography is described in detailed in U.S. Application Publication No. 2008/0064860, the disclosure of which is hereby incorporated by reference. In some embodiments, a wash solution suitable for the invention may contain imidazole or an imidazole derivative. In some embodiments, a suitable wash solution may contain a chaotropic reagent (e.g., urea, sodium thiocynate, and/or guanidinium hydrochloride). In some embodiments, a suitable wash solution may contain a hydrophobic modifier (e.g., organic solvents including ethanol, methanol, propylene glycol, ethylene glycol, hexaethylene glycol, propanol, butanol and isopropanol). In some embodiments, a wash solution suitable for the invention may contain a detergent (e.g., nonionic detergent and/or ionic detergent).

The pH of the wash solution is generally between about 4.5 and about 8.0, for example, 4.5, 5.0, 5.5, 6.0, 6.5, 7.0, 7.5 and 8.0. In same cases, the pH of the wash solution is greater than 5.0 and less than about 8.0, for example, 5.5, 6.0, 6.5, 7.0, 7.5 and 8.0. The wash solution may contain 20 mM to 50 mM Tris (e.g., 20 mM, 30 mM, 40 mM or 50 mM).

The product may be eluted from a washed column, e.g., a Protein A column, by an elution buffer. Typically, a suitable elution buffer may contain Hepes, phosphoric acid, glycine, glycylglycine, one or more organic acids (e.g., acetic acid, citric acid, formic acid, lactic acid, tartaric acid, malic acid, malonic acid, phthalic acid, salicyclic acid), and/or arginine. A suitable elution buffer may further contain a salt (e.g., sodium chloride, potassium chloride, ammonium chloride, sodium acetate, potassium acetate, ammonium acetate, calcium salts, and/or magnesium salts). A suitable salt concentration may range from 0 mM to 1 M (e.g., 0 mM to 500 mM, 0 mM to 100 mM, 0 mM to 50 mM). In some embodiments, a suitable elution buffer contains about 15 mM to about 100 mM NaCl. In some embodiments, NaCl concentration in a elution buffer can be at 4 levels (e.g., 0 mM, 15 mM, 30 mM, and 50 mM). In other embodiments, an elution buffer may contain about 20 mM to about 200 mM arginine or arginine derivatives. In further embodiments, an elution buffer may contain 20 mM to 200 mM glycine. The elution buffer may also contain about 20 mM to about 100 mM HEPES. The elution buffer may also contain about 25 mM to about 100 mM acetic acid. In some embodiments, the elution buffer may contain citric acid (e.g., about 10 mM to about 500 mM citric acid). In some embodiments, the elution buffer may contain glycylglycine (e.g., about 10-50 mM, about 50-100 mM, or about 100-200 mM). In some embodiments, a suitable elution buffer may contain a chaotropic reagent (e.g., urea, sodium thiocynate, and/or guanidinium hydrochloride). In some embodiments, a suitable elution buffer may contain alkyl glycol (e.g., ethylene glycol, propylene glycol, hexaethylene glycol). The pH of the elution buffer may range from about 2.5 to about 4.0. In one embodiment, the pH of the elution buffer is about 3.0.

Eluates from the affinity chromatography columns may be neutralized by neutralization buffers. Suitable neutralization buffers may contain Tris, Hepes, and/or imidazole.

After elution, the affinity chromatography columns may optionally be cleaned, i.e., stripped and/or regenerated, after elution of the protein. This procedure is typically performed regularly to minimize the building up of impurities on the surface of the solid phase and/or to sterilize the matrix to avoid contamination of the product with microorganisms. Stripped and/or regenerated columns may be used repeatedly.

Buffer components may be adjusted according to the knowledge of the person of ordinary skill in the art. Sample buffer composition ranges are provided in the Examples below. Not all of the buffers or steps are necessary, but are provided for illustration only. A high throughput screen, as described in the Examples, may be used to efficiently optimize buffer conditions for Protein A column chromatography.

### Ion Exchange Chromatography

The primary objectives of the ion exchange chromatography step include removal of process-derived impurities (e.g., leached protein A, host cell DNA and/or proteins, adventitious agents) as well as product-related impurities such as HMW aggregates.

In some embodiments, ion exchange chromatography is used in combination with affinity chromatography according to the invention. In some embodiments, ion exchange chromatography (e.g., cation exchange and/or anion exchange chromatography) can be used instead of affinity chromatography.

Various anionic or cationic substituents may be attached to matrices in order to form anionic or cationic supports for chromatography. Anionic exchange substituents include diethylaminoethyl (DEAE), trimethylaminoethyl acrylamide (TMAE), quaternary aminoethyl (QAE) and quaternary amine (Q) groups. Cationic exchange substituents include carboxymethyl (CM), sulfoethyl (SE), sulfopropyl (SP), phosphate (P) and sulfonate (S). Ion exchange resins with polyethyleneimine functional groups, such as POROS® HQ50, are available from Applied Biosystems, Foster City, Calif. Exchange resins with an immobilized recombinant Protein A functional groups, such as POROS® A50, are available from Applied Biosystems, Foster City, Calif. Cellulosic ion exchange resins such as DE23, DE32, DE52, CM-23, CM-32 and CM-52 are available from Whatman Ltd. Maidstone, Kent, U.K. Sephadex-based and cross-linked ion exchangers are also known. For example, CAPTO Q, DEAE-, QAE-, CM-, and SP-Sephadex, and DEAE-, Q-, CM- and S-Sepharose (e.g., DEAE Sepharose FF, Q Sepharose FF and Q Sepharose XL), and Sepharose are all available from GE Healthcare, Piscataway, N.J. Further, both DEAE and CM derivitized ethylene glycol-methacrylate copolymer such as TOYOPEARL™ DEAE-6505 or M, TOYOPEARL™ CM-650S or M, TOYOPEARL™ GIGACAP Q-650, and TOYOPEARL™ GIGACAP CM-650 are available from Toso Haas Co., Philadelphia, Pa. Ion exchange monolithic chromatographic supports, such as CIM®-DISK, may also be used in accordance with the present invention and are available from Bia Separations, Austria. Ion exchange membrane adsorbers, such as Mustang® Q and Mustang® E (Pall Corporation, New York), Sartobind® Q (Sartorius Stedim Biotech S.A., France), and Chromasorb™ (Millipore, Mass.), may also be used in accordance with the present invention.

In some embodiments, an anion exchange column is used. The anion exchange column may be first equilibrated with a high salt buffer and then a low salt buffer before being contacted with proteins. Typically, SMIPs™ bind only weakly to the column, which allows the majority of the product to flow through while impurities with a negative charge, such as host cell DNA and HCPs, bind strongly and are retained. The column may then washed with equilibration buffer to collect additional product weakly bound to the resin. Once the product has been removed from the column, impurities can be stripped using a high salt buffer. The resin can be regenerated, sanitized, and then stored in an ethanol solution.

In some embodiments, it is desirable to use an adsorptive depth filter before the ion exchange chromatography to increase the impurity capacity and life time of resins used in the ion exchange chromatography. For example, Fractogel® EMD TMAE Hi-Cap(M) resin is a strong anion exchanger with a synthetic methacrylate polymeric base. The pores that are formed from intertwined polymer agglomerates have an approximate size of 800 Angstroms. The surface is strongly hydrophilic due to the ether linkages in the polymer. Long, linear polymer chains carry the functional ligands. These polymer chains are known as tentacles. All tentacles are covalently attached to hydroxyl groups of the methacrylate backbone. Additional tentacle resins, such as Fractogel® EMD TMAE (M), Fractogel® EMD TMAE (S), and Fractoprep® TMAE, may also be used in accordance with the present invention. Use of an adsorptive depth pre-filter can protect the TMAE column from impurities in the protein load (e.g., the ProA peak pool). It is likely that these impurities can exhaust or block the binding sites of the TMAE column, reducing resin capacity for impurities. These impurities can be reduced by, for example, prefiltration through an adsorptive depth filter or precipitation of protein.

After elution, the ion exchange chromatography columns may optionally be cleaned, i.e., stripped and/or regenerated, after elution of the protein. This procedure is typically performed regularly to minimize the building up of impurities on the surface of the solid phase and/or to reduce the likelihood of contamination of the product with microorganisms. In some embodiments, ion exchange columns are regenerated by treatment with NaOH solution using concentrations ranging from 10 mM to 2M NaOH. Stripped and/or regenerated columns may be used repeatedly.

As described above, in some embodiments, depth filtration may be used to reduce impurities in a protein preparation. In some embodiments, depth filtration media is a highly porous filter composed of cellulose fibers, diatomaceous earth, and a cationic resin binder. The depth filter can remove impurities by sieving through the cellulose fibers, by hydrophobic adsorption to the diatomaceous earth, and by ionic adsorption to the cationic binder. A depth filter can be, for example, 0.5 cm, 1 cm, 1.5 cm, 2.0 cm thick.

In some embodiments, one or more additives can be added to a protein preparation to induce precipitation and/or enhance protein adsorption to ion exchange columns. In some embodiments, protein precipitation can be induced by additives to reduce the amount of impurities. Various protein precipitation methods are known in the art and can be used in the present invention. For example, proteins can be precipitated by salting out (e.g., using a neutral salt). In some embodiments, proteins can be precipitated by addition of organic solvents (e.g., methanol, ethanol).

In some embodiments, nonionic organic polymers can be used to promote protein binding to surfaces and/or precipitation. Various nonionic organic polymers are commercially available and can be used in the present invention. Examples include, but are not limited to polyethylene glycol (PEG), polypropylene glycol, cellulose, dextran, starch, and polyvinylpyrrolidone. In some embodiments, PEG is used as an additive. PEG with various molecular weight can be used in the present invention. Suitable PEG may have an average polymer molecular weight ranging from, e.g., about 100 to about 20,000 Daltons. In some embodiments, suitable PEG may have an average weight between 200-12,000, 400-20,000,400-1000, 200-1000, 400-2000, 1000-5000, 800-8,000, 1000-10,000, 2,000-12,000 Daltons. In some embodiments, exemplary PEG includes PEG having an average molecular weight of, e.g., 200, 400, 800, 1000, 2,000, 4,000, 6,000, 8000, 10,000, 12,000, 14,000, 16,000, 18,000, 20,000 Daltons, etc. PEG can be added in various concentrations. Lower molecular weight PEGs will generally require a higher concentration to achieve an effect similar to higher molecular weight PEGs. Exemplary suitable PEG concentrations may range from 0-25% (e.g., 0-6%, 0-9%, 0-12%, 0-15%, 0-18%, 0-20%, 3-9%, 3-15%, 6-12%, 6-20%, or 6-25%). PEG or other organic polymers can be linear or branched polymers.

It is contemplated that binding or precipitation effects of PEG are generally dependent on molecular weight of the protein. Typically, PEG effects are greater for larger proteins. For example, lower concentrations of a given molecular weight of PEG are generally used to enhance the binding of larger proteins (e.g., HMW aggregates) as well as viruses compared to concentrations of PEG needed to result in the same amount of enhanced binding of monomeric protein or LMW impurities. Thus, retention of aggregates, complexes, and other large molecule contaminants will generally be enhanced to a greater degree than the unaggregated forms of the proteins from which they are derived. Thus, PEG or other nonionic polymer modification is particular useful for enhanced removal of impurities, in particular those weak binding HMW aggregates, through weak partitioning chromatography. In some embodiments, PEG may be added before anion exchange chromatography but after the affinity chromatography step.

In some embodiments, the use of nonionic organic polymers for protein precipitation can help reduce or eliminate protein denaturation as well as remove detergents and other impurities. In some embodiments, additives (e.g., polyethylene glycols) can be used to concentrate the product.

In some embodiments, the precipitate can be separated by centrifugation, filtration, or other separation methods known in the art. In some embodiments, the precipitate contains contaminants, such as HMW aggregates. In some embodiments, it is desirable to remove contaminant-containing precipitate (e.g., by filtration). In some embodiments, SMIPs™ are present in the precipitate. In some embodiments, it is desirable to dissolve SMIPs™-containing precipitate in a resuspension buffer. In some embodiments, the resuspension buffer has a pH and/or conductivity suitable for direct loading onto an ion exchange column

A high throughput screen, as described in the Examples, may be used to efficiently optimize buffer conditions for ion exchange chromatography.

### Hydroxyapatite Chromatography

The primary objectives of the ceramic hydroxyapatite (cHA) step are the removal of high molecular weight (HMW) aggregates, leached Protein A, additives used to promote precipitation or binding to absorbents (e.g., polyethylene glycol) and host cell-derived impurities, such as DNA and HCPs. cHA resins charged with phosphate around neutral pH and low ionic strength can be used to bind both a monomer protein product (e.g., a SMIP™) and HMW aggregates. Since HMW aggregates bind more tightly to the cHA resins than monomers, the monomers can be selectively eluted using an elution buffer with suitable ionic strength at slightly acidic to slightly basic pH. HMW aggregates can be optionally subsequently washed off the resins using an even higher ionic strength and higher phosphate concentration buffer at neutral pH. As described in the Examples, the present inventors have developed cHA operating conditions that can effectively remove HMW aggregates from protein preparations. In some embodiments, the percentage of HMW aggregates can be reduced from more than 5% (e.g., 5%, 10%, 15%. 20%, 25%, 30%, 35%, 40%, 45%, 50%, 55%, 60%) in a load material to less than 4% (e.g., less than 3.5%, 3.0%, 2.5%, 2.0%, 1.5%, 1.0%, 0.8%, 0.6%, 0.4%, 0.2%, 0.1%) in the purified protein product. In some embodiments, HMW aggregates can be reduced after cHA chromatography, by at least about 2 fold, at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, or at least about 100 fold.

### 1. Hydroxyapatite Resins

Various hydroxyapatite chromatographic resins are available commercially and can be used for the invention. For example, the hydroxyapatite can be in a crystalline form. In some embodiments, hydroxyapatites suitable for the invention may be those that are agglomerated to form particles and sintered at high temperatures into a stable porous ceramic mass. The particle size of the hydroxyapatite may vary widely, but a typical particle size ranges from 1 μm to 1,000 μm in diameter, and may be from 10 μm to 100 μm (e.g., 20 μm, 40 μm, 60 μm, or 80 μm).

A number of chromatographic resins may be employed in the preparation of cHA columns, the most extensively used are Type I and Type II hydroxyapatite. Type I has a high protein binding capacity and better capacity for acidic proteins. Type I is particularly suitable for the purification of small modular immunopharmaceutical proteins. Type II, however, has a lower protein binding capacity, but has better resolution of nucleic acids and certain proteins. The Type II material also has a very low affinity for albumin and is especially suitable for the purification of many species and classes of immunoglobulins. The choice of a particular hydroxyapatite type can be determined by the skilled artisan.

This invention may be used with hydroxyapatite resin that is loose, packed in a column or in a continuous annual chromatograph. In one embodiment of the invention, ceramic hydroxyapatite resin is packed in a column. The choice of column dimensions can be determined by the skilled artisan. In some embodiments, a column diameter of at least 0.5 cm with a bed height of about 20 cm may be used for small scale purification. In some embodiments, a column diameter of from about 35 cm to about 60 cm may be used. In some embodiments, a column diameter of from 60 cm to 85 cm may be used. In some embodiments, a slurry of ceramic hydroxyapatite resin in 200 mM Na₂HPO₄ solution at pH 9.0 may be used to pack the column at a constant flow rate of about 4 cm/min or with gravity.

In some embodiments, the hydroxyapatite resins may optionally be cleaned, i.e., stripped/or and regenerated, after elution of the protein. Stripped and/or regenerated columns can be used repeatedly.

### 2. Operating Buffers and Conditions

Before contacting the hydroxyapatite resin with a load material, it may be important to adjust parameters such as pH, ionic strength, and temperature and in some instances the addition of substances of different kinds Thus, it is an optional step to perform an equilibration of the hydroxyapatite matrix by washing it with a solution (e.g., a buffer for adjusting pH, ionic strength, etc., or for the introduction of a detergent) bringing the necessary characteristics for purification of a protein of interest (e.g., SMIPs™ protein).

In some embodiments, the hydroxyapatite matrix may be equilibrated using a solution containing from 0.01 to 2.0 M NaCl at slightly basic to slightly acidic pH. In some embodiments, an equilibration buffer may contain sodium phosphate, potassium phosphate, and/or lithium phosphate. For example, an equilibration buffer may contain 1 to 20 mM sodium phosphate (e.g., 1 to 10 mM sodium phosphate, 2 to 5 mM sodium phosphate, 2 mM sodium phosphate, or 5 mM sodium phosphate). The equilibration buffer may contain 0.01 to 0.2 M NaCl (e.g., 0.025 to 0.1 M NaCl, 0.05 to 0.2 M NaCl, 0.05 to 0.1 M NaCl, 0.05 M NaCl, or 0.1 M NaCl). The pH of the load buffer may range from 6.2 to 8.0 (e.g., 6.6 to 7.7, 6.5 to 7.5, 6.8, 7.0, 7.1, 7.2, or 7.3). The equilibration buffer may also contain 0 to 200 mM arginine (e.g., 50 mM, 100 mM, 120 mM arginine, 140 mM, 160, or 180 mM arginine). The equilibration buffer may contain 0 to 200 mM HEPES (e.g., 20 mM, 40 mM, 60 mM, 80 mM, 100 mM, 120 mM, 140 mM, 160 mM, 180 mM HEPES). More than one equilibration step may be carried out.

Various protein preparations may be used as load materials (e.g., peak pools from affinity chromatography, flow-through from ion exchange chromatography or raw preparations). In some embodiments, a load may be buffer exchanged into an appropriate loading buffer. For example, a protein preparation may be buffer exchanged into a loading buffer containing 0.2 to 2.5 M NaCl at slightly acidic to slightly basic pH. For example, a loading buffer may contain 1 to 20 mM sodium phosphate (e.g., 2 to 8 mM sodium phosphate, 3 to 7 mM sodium phosphate, or 5 mM sodium phosphate). A loading buffer may contain 0.01 to 0.2 M NaCl (e.g., 0.025 to 0.1 M NaCl, 0.05 to 0.2 M NaCl, 0.05 to 0.1 M NaCl, 0.05 M NaCl, or 0.1 M NaCl). The pH of the loading buffer may range from 6.4 to 7.6 (e.g., from 6.5 to 7.0, or from 6.6 to 7.2).

Loading can be carried out by applying a protein preparation to a packed bed column, a fluidized/expanded bed column containing the solid phase matrix, and/or mixing a protein preparation with hydroxyapatite resins in a simple batch operation where the solid phase matrix is mixed with the solution for a certain time.

After loading, the hydroxyapatite resins can be optionally washed using washing buffer (e.g., a phosphate buffer) to remove loosely bound impurities. Washing buffers that may be employed will depend on the nature of the hydroxyapatite resin and can be determined by one of ordinary skill in the art.

The bound product may be eluted from the column after an optional washing procedure. For effectively eluting monomers of SMIP™ protein from the column, the present invention uses a high ionic strength phosphate buffer at slightly acidic to slightly basic pH. In some embodiments, an elution buffer may contain sodium phosphate, potassium phosphate, and/or lithium phosphate. For example, a suitable elution buffer may contain 1 to 100 mM sodium phosphate (e.g., 2 to 50 mM, 2 to 40 mM, 2 to 35 mM, 2 to 32 mM, 2 to 30 mM, 4 to 35 mM, 4 to 20 mM, 10 to 40 mM, 10 to 35 mM, 4 to 10 mM, or 2 to 6 mM sodium phosphate). In some embodiments, a suitable elution buffer may contain 2 mM, 3 mM, 6 mM, 8 mM, 10 mM, 15 mM, 20 mM, 25 mM, 30 mM, 35 mM, 40 mM, 45 mM, 50 mM, 55 mM, or 60 mM sodium phosphate.

A suitable elution buffer may also contain 0.01 to 2.5 M NaCl (e.g., 0.1 to 2.5 M, 0.1 to 2.0 M, 0.1 to 1.6 M, 0.1 to 1.2 M, 0.1 to 1.0 M, 0.1 to 0.8 M, 0.1 to 0.5 M, 0.2 to 2.5 M, 0.2 to 1.5 M, 0.2 to 1.2 M NaCl, 0.2 to 1.0 M, 0.2 to 0.8 M, 0.3 to 1.1 M, or 0.2 to 0.5 M NaCl). In some embodiments, a suitable elution buffer contains 10 mM, 50 mM, 100 mM, 150 mM, 200 mM, 250 mM, 300 mM, 350 mM, 375 mM, 400 mM, 425 mM, 450 mM, 500 mM, 1.0 M, 1.5 M, 2.0 M, or 2.5 M NaCl).

The pH of a suitable elution buffer may range from 6.4 to 8.5 (e.g., 6.4 to 8.0, 6.4 to 7.8, 6.5 to 7.7, or 6.5 to 7.3). In some embodiments, the pH of a suitable elution buffer may be 6.4, 65, 6.6, 6.8, 7.0, 7.2, 7.4, 7.6, 7.8, 8.0, 8.2, 8.4, or 8.5).

In some embodiments, elution buffers containing varied salt concentrations may be used for elution of the bound product from the column in a continuous or stepwise gradient.

Exemplary buffer and operating conditions are described in the Examples section. High throughput screens, or alternative screens (e.g., gradient elution screens), as described in the Examples, may be used to efficiently optimize buffer and operating conditions for hydroxyapatite chromatography.

Typically, a binding mode cHA chromatography is used for the invention. Alternatively or additionally, a flow-through mode can be used. In flow-through mode, a protein preparation is typically buffer-exchanged into a suitable load buffer as described herein. The protein preparation is then allowed to flow through a hydroxyapatite column, while impurities such as HMW aggregates bind to the column. The column is optionally subsequently washed to allow additional purified protein to flow through the column.

In combination binding/flow-through mode, the protein preparation is allowed to flow through a hydroxyapatite column, with both protein monomer and HMW aggregates binding initially. However, as the loading continues, incoming HMW aggregates are able to bind more tightly than protein monomer and therefore displaces bound monomer. Consequently, the displaced monomer flows through the column. The column is optionally subsequently washed to allow additional displaced monomers to flow through the column.

In addition to the salts and buffers specifically discussed above, chromatography and loading can occur in a variety of buffers and/or salts including sodium, potassium, ammonium, magnesium, calcium, chloride, fluoride, acetate, phosphate, citrate and/or Tris buffers. Specific examples of such buffers and salts are: Tris, sodium phosphate, potassium phosphate, ammonium phosphate, sodium chloride, potassium chloride, ammonium chloride, magnesium chloride, calcium chloride, sodium fluoride, potassium fluoride, ammonium fluoride, calcium fluoride, magnesium fluoride, sodium citrate, potassium citrate, ammonium citrate, magnesium acetate, calcium acetate, sodium acetate, potassium acetate, or ammonium acetate. A high throughput screen, as described in the Examples, may be used to efficiently optimize buffer conditions for cHA chromatography.

In addition, various buffers and solutions described herein may be treated to ensure free of endotoxin and/or exotoxin. In particular, if a purified protein preparation is intended to be used for pharmaceutical and/or clinical purposes, it may be desirable to use endotoxin- and/or exotoxin-free buffers. Various methods to remove endotoxins and/or exotoxins from buffers or solutions are known in the art and can be used in the present invention. For example, buffers and solutions can be depyrogenated. Depyrogenation may be achieved by, e.g., acid-based hydrolysis, oxidation, heat, sodium hydroxide, among others.

### Additional Filtration Steps

Additional membrane filtration steps may be used to reduce adventitious viral and other contaminants, concentrate and/or buffer exchange. Various virus-retaining filters can be used in the present invention including, but not limited to, Planova 20N virus retaining filtration (VRF) and Planova 35N virus retaining filtration (VRF), among others. Various ultrafiltration and/or diafiltration skids (e.g., molecular weight cut-off 10 kDa) can be used to concentrate and/or buffer exchange the process stream in the formulation buffer. The final drug substance can be passed through, e.g., a single-use 0.2 pm filter, to remove any potential adventitious microbial contaminants and particulate material.

### Pharmaceutical Compositions Containing Purified SMIP™ Proteins

The purified protein preparations described herein can be formulated for pharmaceutical uses. In some embodiments, pharmaceutical compositions according to the invention may contain purified SMIP™ proteins with less than 4% (e.g., less than 3.5%, 3.0%, 2.5%, 2.0%, 1.5%, 1.0%, 0.8%, 0.6%, 0.4%, 0.2, 0.1%) HMW aggregates. In some embodiments, pharmaceutical compositions according to the invention may contain purified SMIP™ proteins with more than 70% (e.g., more than 75%, 80%, 85%, 90%, 92%, 94%, 96%, 98%, 99%) of the protein present in a biologically active monomer form.

Pharmaceutical compositions according to the invention may contain one or more pharmaceutically acceptable carriers. Such pharmaceutically acceptable carriers are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified according to the invention and/or known in the art) also can be incorporated into the compositions by any of the methods well known in the art of pharmacy/microbiology.

A pharmaceutical composition of the invention is formulated to be compatible with its intended route of administration. Solutions or suspensions used for administration can include components well known in the art, such as a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, and/or acids or bases, such as hydrochloric acid or sodium hydroxide, among others.

Formulations of the present invention suitable for administration can be in any form known in the art. For example, suitable formulations for oral administration can be capsules, gelatin capsules, sachets, tablets, troches, lozenges, powder, granules, a solution or a suspension in an aqueous liquid or non-aqueous liquid, or an oil-in-water emulsion or a water-in-oil emulsion, among others. The therapeutic can also be administered in the form of a bolus, electuary or paste. As another example, suitable formulations for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). Formulations suitable for intra-articular administration can be in the form of a sterile aqueous preparation of the therapeutic which can be in microcrystalline form, for example, in the form of an aqueous microcrystalline suspension. Liposomal formulations or biodegradable polymer systems can also be used to present the therapeutic for both intra-articular and ophthalmic administration. Formulations suitable for topical administration, including eye treatment, include liquid or semi-liquid preparations such as liniments, lotions, gels, applicants, oil-in-water or water-in-oil emulsions such as creams, ointments or pasts; or solutions or suspensions such as drops. For inhalation treatments, such as for asthma, inhalation of powder (self-propelling or spray formulations) dispensed with a spray can, a nebulizer, or an atomizer can be used. Such formulations can be in the form of a finely comminuted powder for pulmonary administration from a powder inhalation device or self-propelling powder-dispensing formulations [0109] Systemic administration also can be by transmucosal or transdermal means.

According to the present invention, pharmaceutical compositions comprising purified protein preparations can be administered to a mammalian host by any route. Thus, as appropriate, administration can be oral or parenteral, e.g., intravenous, intradermal, inhalation, transdermal (topical), transmucosal, and rectal administration.

The present invention, thus generally described, will be understood more readily by reference to the following examples, which are provided by way of illustration and are not intended to be limiting of the present invention.

### EXAMPLES

### Example 1

### Cell Culture and Harvest

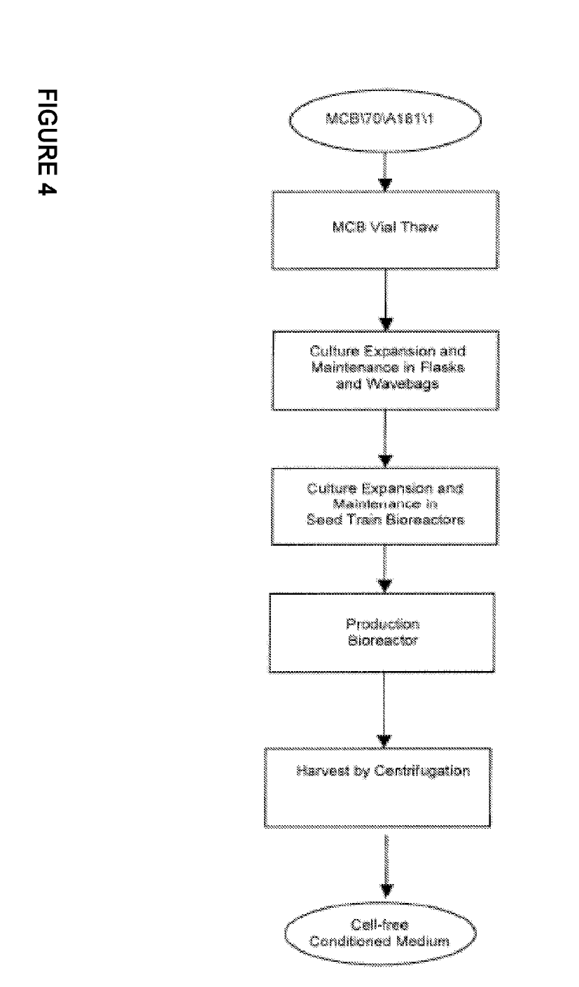

An anti-CD20 SMIP™ protein TRU-015 was produced using a recombinant Chinese Hamster Ovary (CHO) cell line grown in suspension culture. An exemplary cell culture and harvest process for the production of TRU-015 is illustrated in FIG. 4. For all the cell culture steps described herein, liquids added to the step were filtered at least once through a 0.2 μm filter prior to addition. The antifoam suspension, which cannot pass through such filters, was autoclaved prior to addition. Culture broth containing cells was not filtered between steps.

Vials of cells containing CHO cell lines that express TRU-015 were thawed and transferred to culture flasks containing pre-warmed, shake flask medium with 0.45 μM methotrexate for selection pressure.

### Cell Culture Expansion and Maintenance in Flasks and Wavebags

Cell cultures were initially expanded in disposable shake flasks (maximum working volume 1 L) using a batch-refeed process. Each culture flask was incubated with agitation under controlled temperature and CO₂ atmosphere throughout a batch-refeed cycle. At the end of a cycle, all or a portion of the culture was transferred to one or more other shake flasks (or, if there is a sufficient number of cells, to wavebags—see below) and diluted with shake flask medium to a pre-defined target initial cell density. After refeeding, each diluted culture was returned to the incubator, where it was agitated throughout another batch-refeed cycle. All cell culture medium used, up until the step of the terminal fed-batch culture, optionally contained methotrexate to maintain selective pressure.

Culture expansion was continued in wavebags once a sufficient number of cells were obtained by growth in shake flasks. The growth cycle and incubator conditions were the same as for the flasks, except that wavebags were rocked instead of shaken. The wavebag medium was the same as the shake flask medium.

Shake flask and wavebag cultures were continued in this fashion as long as necessary, up until the specified maximum number of generations from the thaw. Typically, a seed train bioreactor was inoculated as soon as a sufficient number of cells were available in the wavebags, and at least one wavebag was maintained as a backup to the seed train bioreactors.

### Cell Culture Expansion and Maintenance in Seed Train Bioreactors

Inoculum expansion was continued in seed train bioreactors. Seed bioreactor medium was added to the bioreactor and supplemented with an autoclaved suspension of antifoam in saline. Inoculum culture from the wavebags was added to a pre-defined target cell density to start each batch-refeed passage. The culture was maintained with agitation under controlled conditions throughout the passage, after which a portion was withdrawn and used to inoculate the next bioreactor, or discarded if not needed. Temperature was controlled at or near 37° C., dissolved oxygen (DO₂) was controlled using sparged 0.2 μm filtered air, oxygen or a blend of both gases, and pH was controlled using carbonate solution (basic titrant).

Each subsequent seed train bioreactor batch-refeed passage began with retention of a portion of the culture from the preceding cycle, dilution with seed bioreactor medium and the addition of antifoam suspension. The seed train bioreactors and wave bags were maintained in batch-refeed operations, both as needed to serve as backups to each other, and to provide inocula for multiple production bioreactor batches. Once a sufficient number of cells were available, the production bioreactor was inoculated.

### Production Bioreactor

Conditioned medium containing TRU-015 were generated in a production bioreactor using a terminal fed-batch process lasting 10 to 15 days. Inoculum culture from a seed train bioreactor was added to initial production medium in the production bioreactor. An antifoam suspension was added. The resulting culture was maintained with agitation under controlled conditions throughout the batch. Approximately 4 days after inoculation, the temperature set point was shifted from 37° C. to 31° C. Throughout the production cell culture process, DO₂ was controlled using sparged 0.2 μm filtered air, oxygen or a blend, and pH was controlled using carbonate solution (basic titrant). A concentrated feed medium solution was also added during the fed-batch. Between 10 and 15 days after inoculation, the entire volume of the production bioreactor culture was harvested. The harvest day was chosen based on schedule considerations and/or on culture viability considerations.

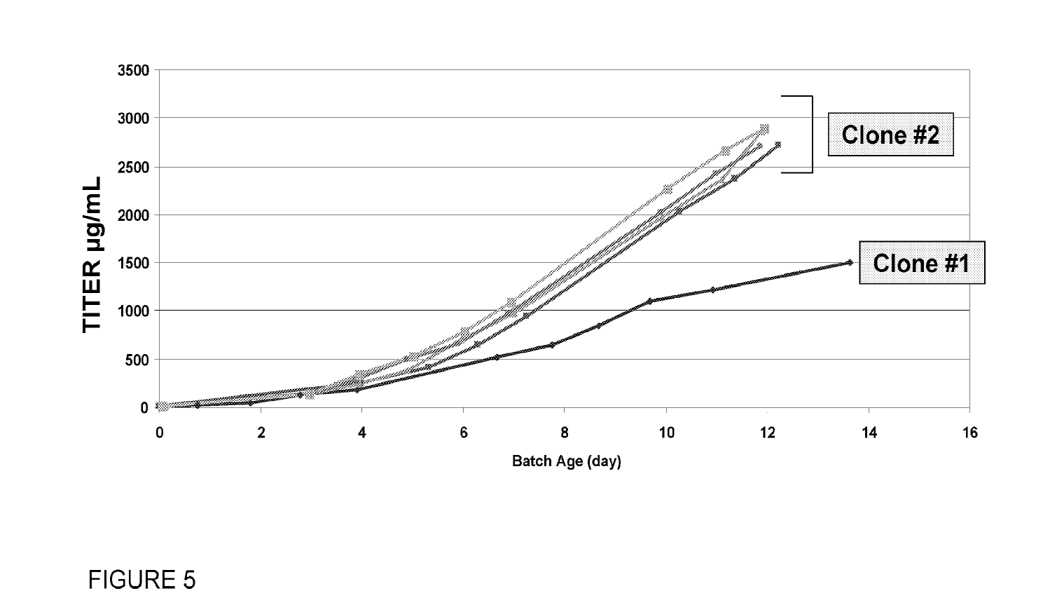

FIG. 5 shows an exemplary daily titer measurements (μg/ml) of the production bioreactor of TRU-015 produced by two different CHO cell clones over a 14 day culture period. Peak titer values were obtained between days 12 and 14 of production bioreactor growth. Peak titer values ranged from 1500 to 3000 μg/ml.

### Harvest by Disc Stack Centrifugation (DSC)

Conditioned medium from the production bioreactor was harvested through a disc-stacked centrifuge to yield clarified conditioned medium (CCM). One objective of the DSC step was to separate CHO cells and cell debris from conditioned medium containing the SMIP™ protein. The contents of the bioreactor vessel were fed via pressure through the DSC, then a pad filtration apparatus, and then 0.2 μm filters into a filtrate vessel or bag. Upon completion of the harvest processing, a HEPES/EDTA buffer solution was spiked into the filtered centrate pool. One purpose of this spike was to reduce the generation of acidic species during the in-process hold period between the DSC and subsequent steps. EDTA may also inhibit protease activities and reduce protein A leaching. The DSC step was operated at room temperature.

### Example 2

### High Throughput Screening of Chromatography Conditions

High throughput screens were used to develop optimal conditions for purification process. Early high throughput screening of potential chromatography options allows rapid identification of operating windows. Comparison of high throughput screening results to database further narrows operating conditions. High throughput screening minimizes the number of column runs and in-process materials required and enables parallel development efforts.

### Protein A Chromatography

The primary objectives of the Protein A chromatography step include product capture from cell-free clarified conditioned medium and separation of TRU-015 from process-derived impurities (e.g., host cell DNA and host cell proteins [HCPs], medium components, and adventitious agents).

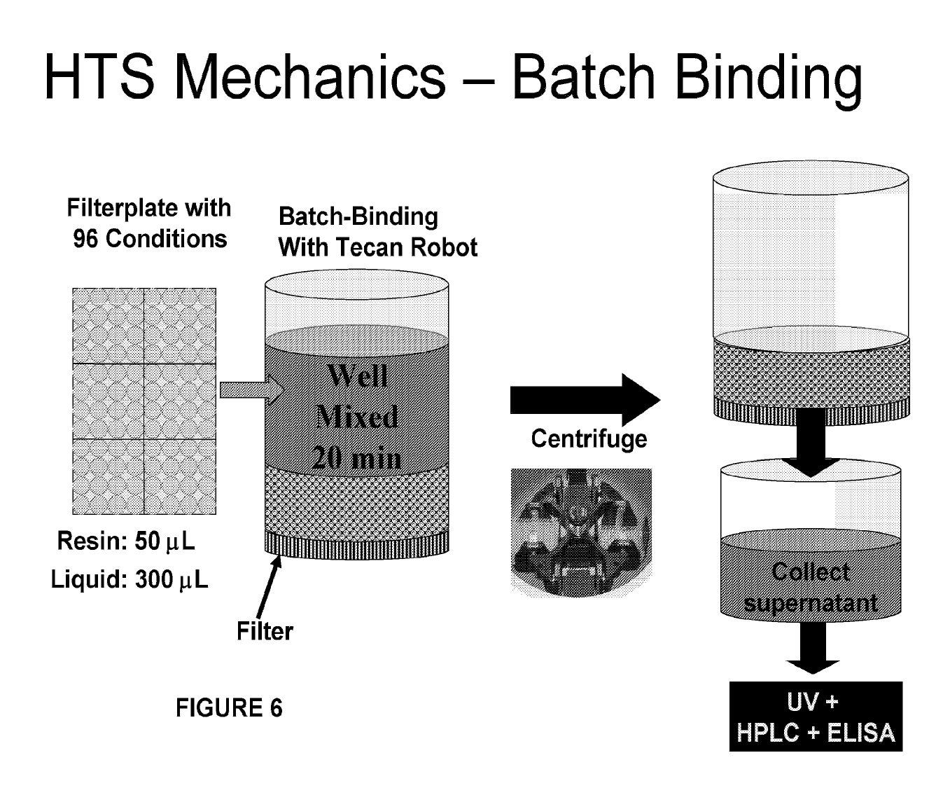

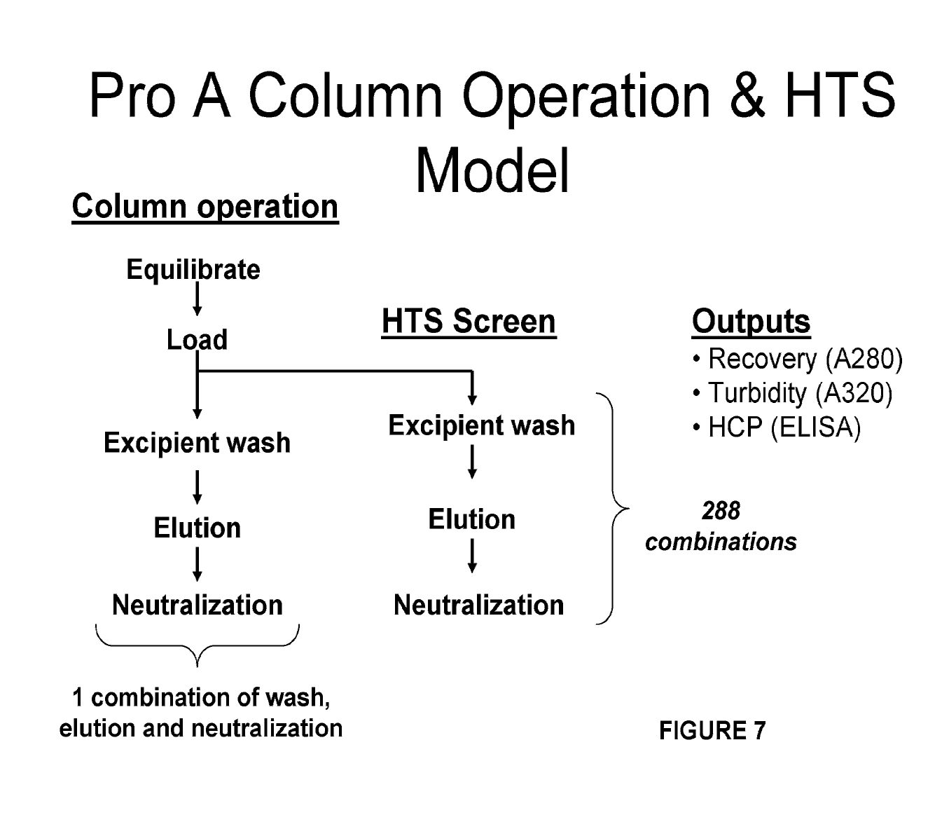

A high throughput screen was performed to optimize the Protein A column conditions to increase product capture, impurity removal and minimize eluate precipitations. An exemplary design of a high throughput screen using a batch binding mechanism is illustrated in FIG. 6 and an exemplary of Protein A column operation and high throughput screen model is illustrated in FIG. 7. As shown, different combinations of excipient wash, elution and neutralization conditions were screened. In particular, the screen at least varied levels of sodium chloride, calcium chloride, arginine, and Tris as wash excipients; HEPES, acetic acid, and glycine as elution buffers; Tris, HEPES, and imidazole as neutralization buffers; and sodium chloride concentration levels in elution (e.g., 0 mM, 15 mM, 30 mM, and 50 mM).

The screen used filterplates containing 96 wells with each well having a different condition. Each well contained about 50 μl of resin and 300 μl of liquid. The resin and liquid was mixed for about 20 minutes using Tecan Robot (Tecan US, Inc. 4022 Stirrup Creek Drive Suite 310 Durham, N.C. 27703, USA) and the plates were centrifuged to collect supernatant. The supernatant from each well was analyzed to determine the recovery of the product, the amount of monomer and aggregates, and the presence of host cell proteins. For example, UV absorbance at A280 was used to determine the overall protein concentration. The turbidity was measured by absorbance at A320. The amount of monomer and aggregates was measured by size exclusive HPLC. The host cell protein was characterized by ELISA.

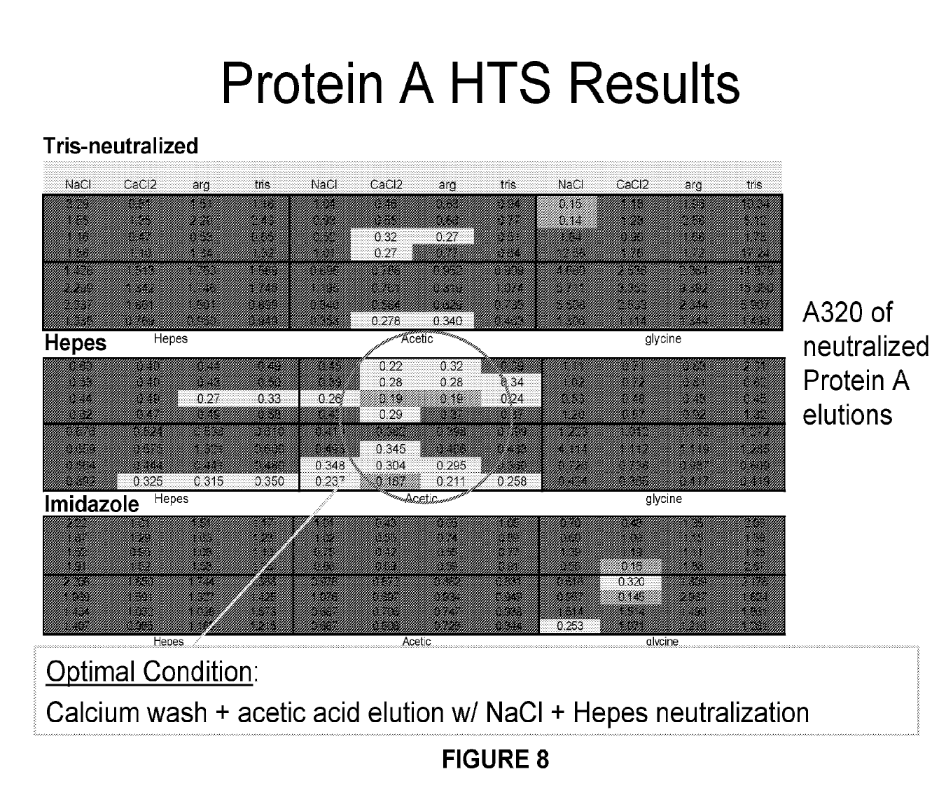

Exemplary Protein A high throughput screen results are shown in FIG. 8. One exemplary favorable condition identified in this experiment included calcium wash, acetic acid elution with sodium chloride, and HEPES neutralization (see FIG. 8).

### Ion Exchange Chromatography

The primary objectives of ion exchange chromatography include removal of process-derived impurities (e.g., leached Protein A, host cell DNA and proteins, and adventitious agents) as well as product-related impurities such as high molecular weight (HMW) species. Similarly, high throughput screen was used to identify potential operating conditions for anion exchange chromatography (AEX) conditions and cation exchange chromatography (CEX) to remove impurities. Exemplary variables tested are shown in Table 1.

TABLE 1
High Throughput Screen Approach for AEX and CEX

| | | # of Resins | | |
|---|---|---|---|---|
| Variable | Resin Type | Tested | pH | Ionic Strength |
| Typical Range | AEX | 1 | 7.0-8.75 | 10-210 mM |
| | | | (8 levels) | (12 levels) |
| | CEX | 2 | 4.5-6.5 | 20-300 mM |
| | | | (8 levels) | (6 levels) |

Ceramic Hydroxyapatite (cHA) Chromatography

The primary objectives of ceramic hydroxyapatite (cHA) chromatography include the removal of high molecular weight (HMW) aggregates, leached Protein A, and host cell-derived impurities, such as DNA and HCPs.

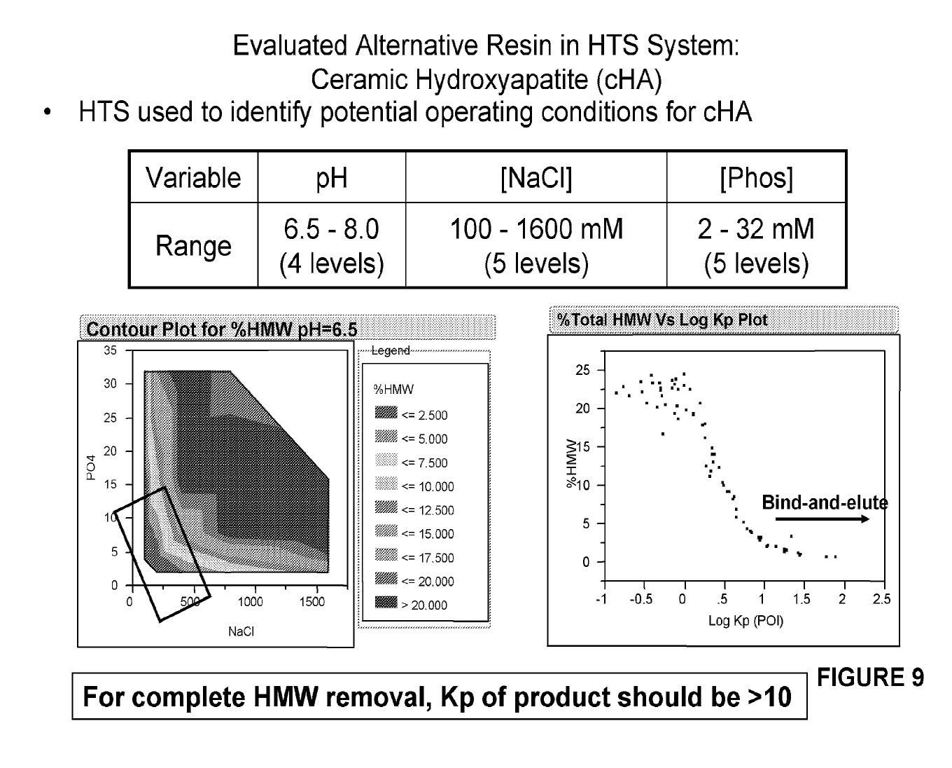

High throughput screens were also performed to optimize operating conditions for ceramic hydroxyapatite chromatography. The screens at least varied pH, salts concentrations and phosphate concentration. Exemplary variables are shown in FIG. 9. Exemplary results with respect to the removal of HMW aggregate are also shown in FIG. 9.

### Example 3

### Development of the cHA Chromatography Step

A high throughput screen was able to qualitatively predict suitable monomer recovery and HMW aggregates removal conditions in a column purification scheme. For example, the high throughput screens identified that the cHA chromatography step was effective in removing HMW aggregates. Approximate ranges of salt or buffer conditions suitable for removing HMW aggregates were also predicted (see FIG. 9). Alternative screens may be used to further refine the conditions identified by high throughput screens.

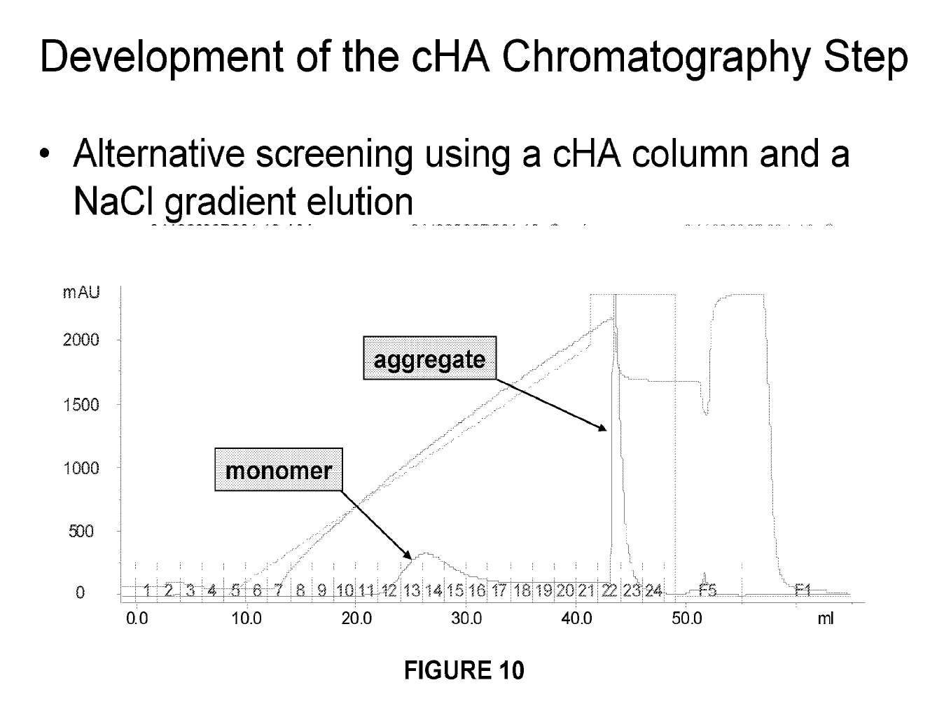

An alternative screening using a cHA column and a sodium chloride gradient elution was performed. An exemplary scheme and result is shown in FIG. 10.

Potential elution buffers based on high throughput and alternative screenings were further evaluated in the step-elution mode. For example, a Protein A column peak pool with 60% HMW aggregates 6000 ppm HCP was purified using cHA columns with different combinations of phosphate and NaCl concentrations as shown in Table 2.

TABLE 2
Exemplary Purification of Protein A Column Peak Pool

| | Phos. | NaCl | | | HCP | ProA |
|---|---|---|---|---|---|---|
| Run | (mM) | (mM) | % HMW | % REC | (ppm) | (ppm) |
| 1 | 30 | 50 | 0.2 | 88 | 954 | <1 |
| 2 | 35 | 50 | 0.2 | 92 | 1529 | <1 |
| 3 | 4 | 350 | 0.4 | 88 | 50 | <1 |
| 4 | 4 | 375 | 0.5 | 86 | 42 | <1 |

An anion exchange chromatography pool with 59% HMW and 290 ppm HCP was purified using cHA columns with different combinations of phosphate and NaCl concentrations as shown in Table 3.

TABLE 3
Exemplary Purification of Anion Exchange
Chromatography Pool

| | Phos | NaCl | | | HCP | ProA |
|---|---|---|---|---|---|---|
| Run | (mM) | (mM) | % HMW | % REC | (ppm) | (ppm) |
| 1 | 60 | 10 | 0.1 | 73 | 137 | <1 |
| 2 | 40 | 50 | 0.5 | 80 | 198 | <1 |
| 3 | 20 | 100 | 0.5 | 78 | 101 | <1 |
| 4 | 10 | 200 | 1 | 82 | 32 | <1 |

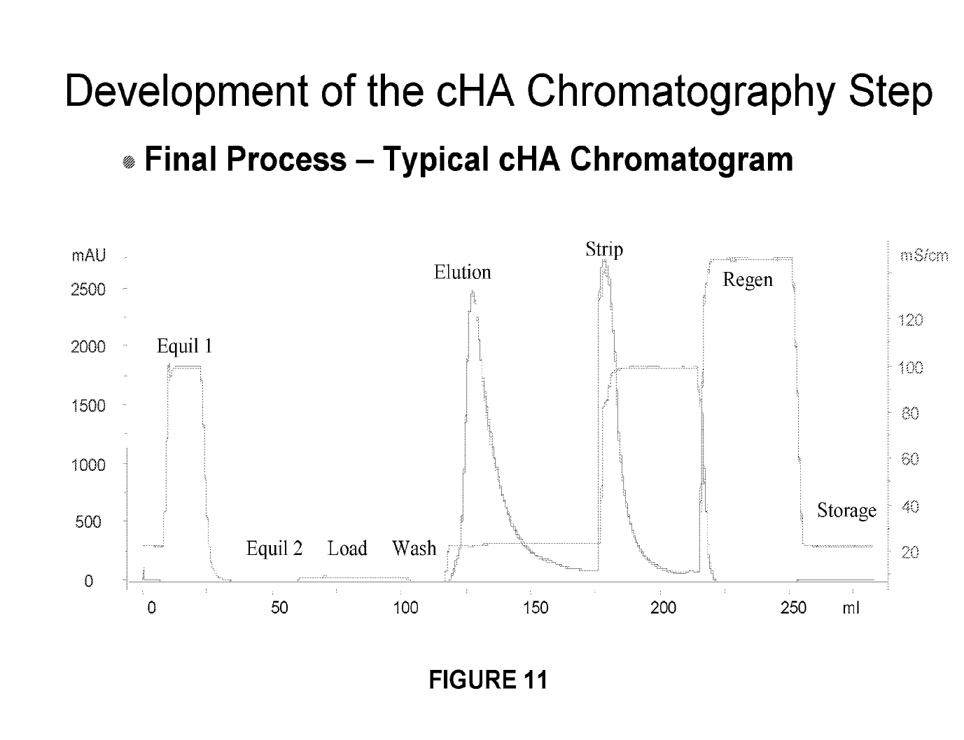

An exemplary typical cHA chromatography step developed based on the experiments described herein is shown in FIG. 11.

### Example 4

### Purification Process for TRU-015

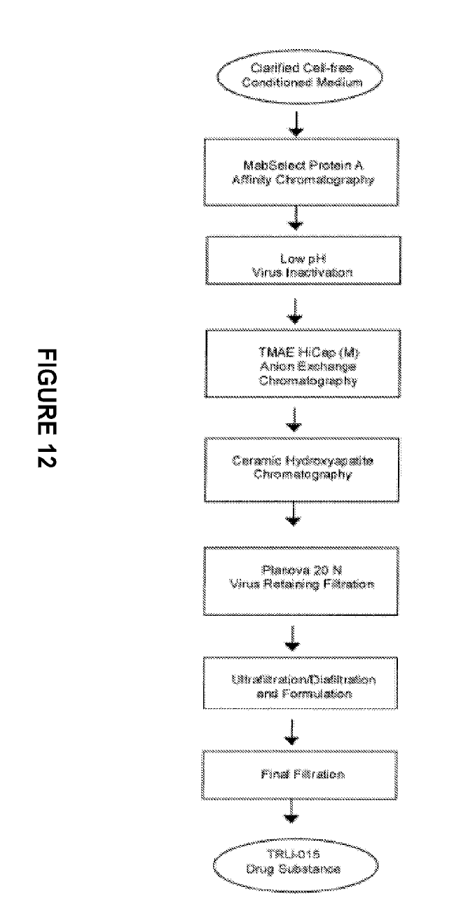

Based on the experiments described above, purification processes of SMIP™ proteins have been developed. An exemplary purification process of TRU-015 is illustrated in FIG. 12. This process includes three chromatographic steps and three membrane filtration steps. All steps were performed at room temperature unless indicated otherwise.

Clarified cell-free conditioned medium prepared as described in Example 1 was first subject to MabSelect™ Protein A affinity chromatography. A 17.7 L (30 cm diameter×25 cm height) MabSelect™ column with recombinant Protein A resin (GE Healthcare, Piscataway, N.J.) was used. The Protein A column was equilibrated with Hepes-buffered saline and loaded with clarified conditioned medium. The loaded resin was washed with a Hepes buffer containing calcium chloride to further reduce the level of impurities, followed by a wash containing a low concentration of Hepes buffer and sodium chloride. The bound product was eluted from the column with a low pH acetic acid buffer.

The product pool was held at pH≦4.1 for about 1.5±0.5 hours at 18° C. to 24° C. The low pH hold was designed to inactivate enveloped viruses. The elution pool was then neutralized with a concentrated Hepes buffer. The resin was regenerated, sanitized, and then stored in an ethanol solution.

A TMAE HiCap (M) column was equilibrated first with a high salt buffer and then a low salt buffer. The column was loaded with the neutralized MabSelect rProtein A peak. One or multiple neutralized MabSelect rProtein A peak pools were loaded onto the TMAE HiCap (M) column. TRU-015 binds only weakly to the column, which allows the majority of the product to flow through while impurities with a negative charge, such as host cell DNA and HCPs, bind strongly and are retained. The TMAE HiCap (M) column was then washed with equilibration buffer to collect additional product weakly bound to the resin. Once the product was removed from the TMAE HiCap (M) column, impurities were stripped using a high salt buffer. The resin was regenerated, sanitized, and then stored in an ethanol solution.

A cHA column was first equilibrated with a high salt buffer and then followed with a low salt buffer. The TMAE flow-through pool was then applied to the cHA column. After loading, the column was washed with the low salt equilibration buffer and TRU-015 was recovered using a higher salt buffer (see Example 3). The HMW species and other impurities were removed from the column at much higher salt and phosphate concentrations. The column was regenerated and then stored in a sodium hydroxide solution.

A Planova 20N virus retaining filtration (VRF) step provides a significant level of viral clearance for assurance of product safety by removal of particles that may represent potential adventitious viral contaminants. The single-use Planova 20N VRF device was equilibrated and loaded with the cHA product pool. The TRU-015 protein was collected in the permeate stream. After the load was processed, an equilibration buffer flush was used to recover additional product remaining in the system.

An ultrafiltration/diafiltration skid (molecular weight cut-off 10 kDa) was used to concentrate and buffer exchange the process stream in the formulation buffer. After equilibration, the load solution was initially concentrated approximately 8-fold and then diafiltered at least 10-fold with formulation buffer (e.g., 20 mM L-histidine, 4% mannitol, 1% sucrose, pH 6.0). Following further concentration, the pool was recovered from the system with a formulation buffer flush.

The drug substance was passed through a single-use 0.2 μm filter to remove any potential adventitious microbial contaminants and particulate material.

Filtered TRU-015 drug substance was filled into, e.g., stainless steel vessels, frozen, and stored at −50° C.±10° C.

The column performance at each step was analyzed to determine the product recovery and impurity removal efficiency. Exemplary results are summarized in Table 4.

TABLE 4
Exemplary summary of column performance (lab-scale)

| | | | ProA | % Rec | % Rec |
|---|---|---|---|---|---|
| Step | % HMW | HCP (ppm) | (ppm) | (POI)* | (total) |
| Harvest | 50-61 | 300,000 | NA | 95 | 95 |
| Protein A | 50-61 | 5,000-45,000 | 20-50 | 85-95 | 81-90 |
| AEX | 48-60 | 50-400 | 1-3 | 85-95 | 86-92 |
| cHA | <2 | <100 | <1 | 75-85 | 35-41 |

| | | |
|---|---|---|
| Total Process Recovery excluding VRF and UF/DF: | 51-73 | 23-32 |

*POI: Product-of-Interest

As shown in Table 4, the cHA chromatography step effectively removed most of the HMW aggregates. The total process recovery for the product of interest ranged from 51-73% and the product yield was about 23-32%. The results shown in Table 4 were based on lab-scale purification processes. Comparable results were obtained for clinical manufacturing processes. For example, based on multiple clinical-scale processes, the average yield was about 28%, and the percentage of HMW aggregates in purified product was about 0.8% or less (reduced from 50-60% in the starting material). Compared to an existing process, there is more than an 8-fold increase in productivity due to increases in protein expression/harvest and purification yield.

### Example 5

### Pad Filtration of Load Material

Load materials can be optionally subjected to pad filtration to increase the capacity for impurities and life time of the anion exchange column. For example, the TMAE Hi-cap resin is a strong anion exchanger with a synthetic methacrylate polymeric base. The pores that are formed from intertwined polymer agglomerates have an approximate size of 800 Angstroms. The surface is strongly hydrophilic due to the ether linkages in the polymer. Long, linear polymer chains known as tentacles, carry the functional ligands. The tentacles are covalently attached to hydroxyl groups of the methacrylate backbone. Impurities can exhaust or block the binding surface of the TMAE column reducing capacity.

Use of an adsorptive depth pre-filter can protect a column, such as the TMAE column, from the impurities in the load material (e.g., MabSelect™ ProA peak pool). Depth filtration media is typically a highly porous one cm thick filter composed of cellulose fibers, diatomaceous earth, and a cationic resin binder. The depth filter can remove impurities by sieving through the cellulose fibers, by hydrophobic adsorption to the diatomaceous earth, and by ionic adsorption to the cationic binder.

During the purification of TRU-015, a Millistack A1HC PAD filter (Millipore, Billerica, Mass.) was used to remove impurities from the MabSelect ProA peak pool before loading to the TMAE column and improved the TMAE capacity by at least 2-fold. For example, prefiltration of the product load with the adsorptive depth filter Millistack AIHC provided an increase in the load challenge on a subsequent TMAE Hi-Cap resin column from 100 to 200 mg/mL, as indicated by the secondary breakthrough of contaminants. The AIHC was run at a flux of 200 liters per square meter per hour and loaded to 200 liters per square meter.

### Example 6

### Capture of SBI-087 Using Cation Exchange Chromatography

This example demonstrates that CEX can be used to effectively capture SMIP™ instead of affinity chromatography. It is contemplated that using CEX may have various benefits including lower capture costs, possibly greater capacities than certain affinity columns, solid potential for HMW reduction and potential elimination of cHA or anion exchange step, among others.

SBI-087 was used in this experiment. Load material for CEX can be acidified using any suitable acid. Exemplary acidification conditions are shown in Table 5.

TABLE 5
Exemplary summary of load material acidification

| | Percent 1M Acetic |
|---|---|
| Load pH | Acid addition (v/v) |
| 4.25 | 20% |
| 4.5 | 12% |
| 4.75 | 7% |
| 5 | 5% |

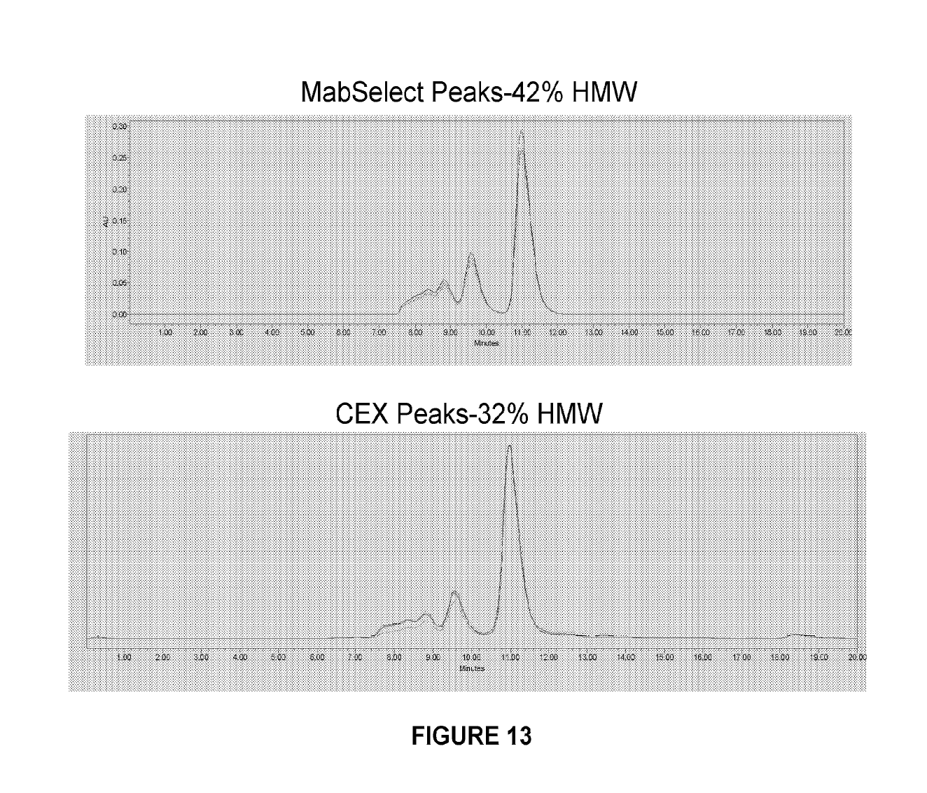

Reduction of amount of HMW aggregates by MabSelect™ Protein A affinity chromatography was compared to that by CEX using a batch-binding method. As shown in FIG. 13, the removal of HMW aggregates by CEX is comparable or better than MabSelect™ affinity chromatography, indicating that CEX can be used to replace affinity chromatography for removal of HMW aggregates from a protein preparation.

Exemplary CEX steps include load and elute. Operating conditions for CEX capture were optimized using high throughput screen methods. For example, two types of CEX resins, GigaCap® and Capto™ S, were used. Load challenge of 25 mg/mLr vs. 75 mg/mLr were used. Load pH (adjusted with 1M acetic acid) was 4.25, 4.5, 4.75, or 5.0. Elution conditions were as follows:

Elution (total mM Na+):

pH 5 (acetate): 100, 125, 150, 175 mM

pH 6.5 (MES): 40, 65, 90, 115

pH 8.0 (Tris): 20, 40, 60, 80

Assumes 50 mM buffer

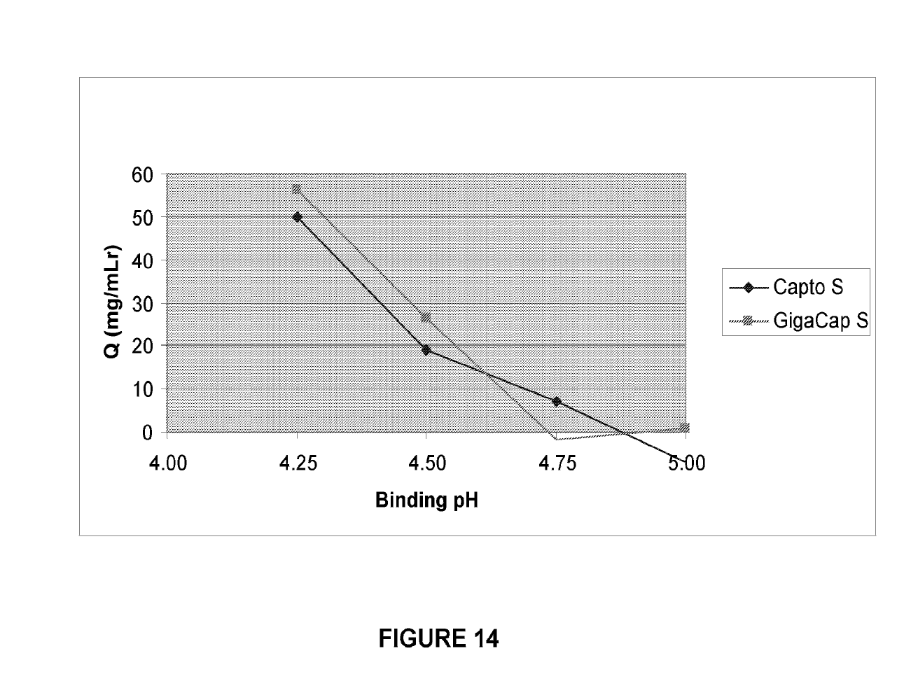

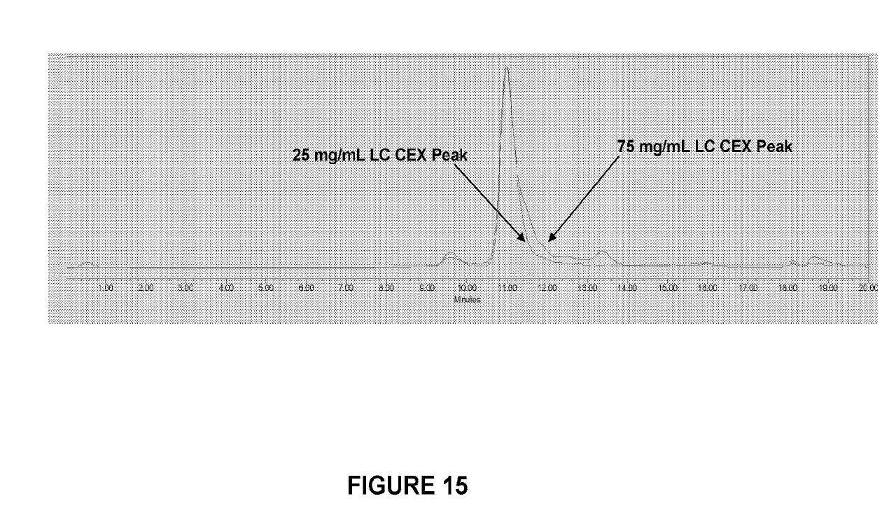

Exemplary results showing binding capacities with 2 hour incubation at room temperature are shown in FIG. 14. It was also observed that higher or longer challenge can lead to more LMW species in the eluted pool. Exemplary results illustrating CEX peaks eluted from columns loaded with 25 vs. 75 mg/mLr LC are shown in FIG. 15. Exemplary strip conditions utilized were 8M urea, 2M NaCl, pH 6. CEX resins can be stripped and/or reused.

### Example 7

### Removal of HMW Using Anion Exchange Chromatography

This example demonstrates that anion exchange chromatography can be used to effectively remove HMW impurities from SMIP™ preparation. In this experiment, SBI-087 was used as an exemplary SMIP™ protein.

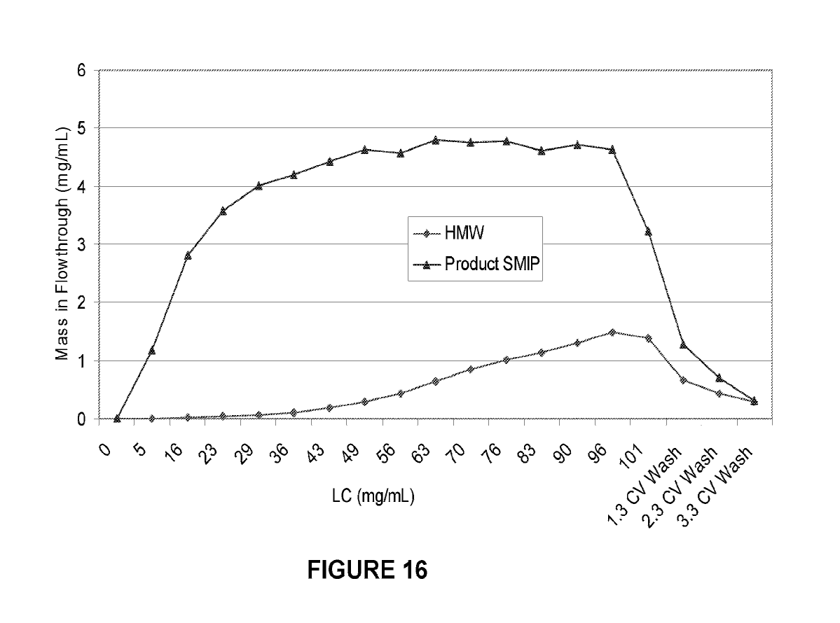

An anion exchange chromatography step was developed using the high throughput screen approach as outlined in Table 1. The chromatography conditions derived from that screen were employed in an exemplary run using a load containing Protein A peak pool with 37% HMW. A packed column of Fractogel® TMAE HiCap was run in the weak partitioning chromatography mode to a load challenge of 100 and 93 mg/mL, respectively. FIG. 16 shows an exemplary effective removal of HMW. The collected pool was 88% pure with >95% yield of the “monomeric” SMIP™. The post-load wash allowed for greater recovery of “monomeric” SMIP™ protein.

These results demonstrated that the second column (e.g., AEX) chromatography can effect substantial removal of HMW, which allows for more flexibility and lower costs in developing and running the downstream cHA step. In addition, these results also indicate that it is feasible to develop a 2-column (e.g., protein A to AEX) process to remove substantial amount of HMW impurities from protein preparation.

EXEMPLARY SMIP ™ SEQUENCES
Italics: Linker sequence
Underline: CDR sequences

| Construct Name | | | |
|---|---|---|---|
| | VK3 | VH5 | |
| 18011 | 2Lm19-3 | 2H5m3 | EIVLTQSPATLSLSPGERATLSCRASQSVSYIV |
| | | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCAR |
| | | | SYYSNSYWYFDLWGRGTLVTVSS |
| | | | (SEQ ID NO: 1) |
| 18008 | 2Lm5 | 2H5 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGTGEVQLVQSGA |
| | | | EVKKPGESLKISCKGSGYSFTSYNMHWVRQMP |
| | | | GKGLEWMGAIYPGNGDTSYNQKFKGQVTISA |
| | | | DKSISTAYLQWSSLKASDTAMYYCAR |
| | | | VVYYSNSYWYFDLWGRGTLVTVSS |
| | | | (SEQ ID NO: 2) |
| 18010 | 2Lm19-3 | 2H5 | EIVLTQSPATLSLSPGERATLSCRASQSVSYIV |
| | | | WYQQKPGQAPRLLIYAPSNLASGIPARFSGSG |
| | | | SGTDFTLTISSLEPEDFAVYYCQQWSFNPPTF |
| | | | GQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCAR |
| | | | VVYYSNSYWYFDLWGRGTLVTVSS |
| | | | (SEQ ID NO: 3) |
| 18009 | 2Lm5 | 2H5m3 | EIVLTQSPATLSLSPGERATLSCRASQSVSYIV |
| | | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCA |
| | | | RVVYYSNSYWYFDLWGRGTLVTVSS |
| | | | (SEQ ID NO: 4) |
| 2Lm5 2H3m3 | 2Lm5 | 2H3m3 | EIVLTQSPATLSLSPGERATLSCRASQSVSSYMH |
| | | | WYQQKPGQAPRLLIYAPSNLASGIPARFSGSGS |
| | | | GTDFTLTISSLEPEDFAVYYCQQWSFNPPTFG |
| | | | QGTKVEIKDGGGSGGGGSGGGGTGEVQLLES |
| | | | GGGLVQPGGSLRLSCAASGFTFSSYNMHWVR |
| | | | QAPGKGLEWVSAIYPGNGDTSYNQKFKGRFT |
| | | | ISRDNSKNTLYLQMNSLRAEDTAVYYCA |
| | | | KSYYSNSYWYFDLWGRGTLVTVSS |
| | | | (SEQ ID NO: 5) |

Construct Name

| VK3 | VH1 | |
|---|---|---|
| 2L | 2Hm | EIVLTQSPATLSLSPGERATLSCRASSSVSSYMHW |
| | | YQQKPGQAPRLLIYAPSNLASGIPARFSGSGSGTD |
| | | FTLTISSLEPEDFAVYYCQQWSFNPPTFGQGTKV |
| | | EIKDGGGSGGGGSGGGGSSQVQLVQSGAEVKKP |
| | | GASVKVSCKASGYTFTSYNMHWVRQAPGQGLE |
| | | WMGAIYPGNGDTSYNQKFKGRVTMTRDTSTST |
| | | VYMELSSLRSEDTAVYYCARSVYYSN.YWYFDL |
| | | WGRGTLVTVSS |
| | | (SEQ ID NO: 6) |
| 2Lm | 2Hm | EIVLTQSPATLSLSPGERATLSCRASSSVSYMIW |
| | | YQQKPGQAPRLLIYAISNLASGIPARFSGSGSGT |
| | | DFTLTISSLEPEDFAVYYCQQWISNPPTFGQGTK |
| | | VEIKDGGGSGGGGSGGGGSSQVQLVQSGAEVK |
| | | KPGASVKVSCKASGYTFTSYNMHWVRQAPGQ |
| | | GLEWMGAIYPGNGDTSYNQKFKGRVTMTRDT |
| | | STSTVYMELSSLRSEDTAVYYCAR |
| | | SVYYSN.YWYFDLWGRGTLVTVSS |
| | | (SEQ ID NO: 7) |
| 2Lm | 2H | EIVLTQSPATLSLSPGERATLSCRASSSVSYMIW |
| | | YQQKPGQAPRLLIYAISNLASGIPARFSGSGSGT |
| | | DFTLTISSLEPEDFAVYYCQQWISNPPTFGQGTK |
| | | VEIKDGGGSGGGGSGGGGSSQVQLVQSGAEVK |
| | | KPGASVKVSCKASGYTFTSYNMHWVRQAPGQ |
| | | GLEWMGAIYPGNGDTSYNQKFKGRVTMTRDT |
| | | STSTVYMELSSLRSEDTAVYYCAR |
| | | VVYYSNSYWYFDLWGRGTLVTVSS |
| | | (SEQ ID NO: 8) |
| 2Lm1 | 2Hm | EIVLTQSPATLSLSPGERATLSCRASQSSVSYMH |
| | | WYQQKPGQAPRLLIYAPSNLASGIPARFSGSGS |
| | | GTDFTLTISSLEPEDFAVYYCQQWISNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTRD |
| | | TSTSTVYMELSSLRSEDTAVYYCAR |
| | | SVYYSN.YWYFDLWGRGTLVTVSS |
| | | (SEQ ID NO: 9) |
| 2Lml | 2H | EIVLTQSPATLSLSPGERATLSCRASQSSVSYMH |
| | | WYQQKPGQAPRLLIYAPSNLASGIPARFSGSGSG |
| | | TDFTLTISSLEPEDFAVYYCQQWISNPPTFGQGTK |
| | | VEIKDGGGSGGGGSGGGGSSQVQLVQSGAEVKKP |
| | | GASVKVSCKASGYTFTSYNMHWVRQAPGQGLEW |
| | | MGAIYPGNGDTSYNQKFKGRVTMTRDTSTSTVY |
| | | MELSSLRSEDTAVYYCARVVYYSNSYWYFDLW |
| | | GRGTLVTVSS |
| | | (SEQ ID NO: 10) |
| 2Lm2 | 2Hm | EIVLTQSPATLSLSPGERATLSCRASQSVSYMIW |
| | | YQQKPGQAPRLLIYAISNLASGIPARFSGSGSGT |
| | | DFTLTISSLEPEDFAVYYCQQWSFNPPTFGQGTK |
| | | VEIKDGGGSGGGGSGGGGSSQVQLVQSGAEVK |
| | | KPGASVKVSCKASGYTFTSYNMHWVRQA |
| | | PGQGLEWMGAIYPGNGDTSYNQKFKGRV |
| | | TMTRDTSTSTVYMELSSLRSEDTAVYYCA |
| | | RSVYYSN.YWYFDLWGRGTLVTVSS |
| | | (SEQ ID NO: 11) |
| 2Lm3 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWTSNPPTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCA |
| | | RSVYYSN.YWYFDLWGRGTLVTVSS |
| | | (SEQ ID NO: 12) |
| 2Lm4 | 2Hm | EIVLTQSPATLSLSPGERATLSCRASQSVSSYMH |
| | | WYQQKPGQAPRLLIYAPSNLASGIPARFSGSGS |
| | | GTDFTLTISSLEPEDFAVYYCQQWTSNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | SVYYSN.YWYFDLWGRGTLVTVSS |
| | | (SEQ ID NO: 13) |
| 2Lm5 | 2Hm | EIVLTQSPATLSLSPGERATLSCRASQSVSYMH |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGRV |
| | | TMTRDTSTSTVYMELSSLRSEDTAVYYCA |
| | | RSVYYSN.YWYFDLWGRGTLVTVSS |
| | | (SEQ ID NO: 14) |
| 2Lm5-1 | 2Hm3 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | S.YYSNSYWYFDLWGRGTLVTVSS |
| | | (SEQ ID NO: 15) |
| 2Lm5-2 | 2Hm4 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | V.YYSNSYWYFDLWGRGTLVTVSS |
| | | (SEQ ID NO: 16) |
| 2Lm5-3 | 2Hm5 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | SVYY.NSYWYFDLWGRGTLVTVSS |
| | | (SEQ ID NO: 17) |
| 2Lm6 | 2Hm | EIVLTQSPATLSLSPGERATLSCRASQSVSYMH |
| | | WYQQKPGQAPRLLIYAPSNLASGIPARFSGSG |
| | | SGTDFTLTISSLEPEDFAVYYCQQWTSNPPTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCAR |
| | | SVYYSN.YWYFDLWGRGTLVTVSS |
| | | (SEQ ID NO: 18) |
| 2Lm6-1 | 2Hm3 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWTSNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | S.YYSNSYWYFDLWGRGTLVTVSS |
| | | (SEQ ID NO: 19) |
| 2Lm6-2 | 2Hm4 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWTSNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | V.YYSNSYWYFDLWGRGTLVTVSS |
| | | (SEQ ID NO: 20) |
| 2Lm6-3 | 2Hm5 | EIVLTQSPATLSLSPGERATLSCRASQSVSYMH |
| | | WYQQKPGQAPRLLIYAPSNLASGIPARFSGSG |
| | | SGTDFTLTISSLEPEDFAVYYCQQWTSNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYY.NSYWYFDLWGRGTLVTVS S |
| | | (SEQ ID NO: 21) |
| 2Lm7 | 2Hm | EIVLTQSPATLSLSPGERATLSCRASSSVSYMH |
| | | WYQQKPGQAPRLLIYATSNLASGIPARFSGSG |
| | | SGTDFTLTISSLEPEDFAVYYCQQWTSNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 22) |
| 2Lm8 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPYTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCA |
| | | RSVYYSN.YWYFDLWGRGTLVTVSS |
| | | (SEQ ID NO: 23) |
| 2Lm9 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPFTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 24) |
| 2Lm10 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPLTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCA |
| | | RSVYYSN.YWYFDLWGRGTLVTVSS |
| | | (SEQ ID NO: 25) |
| 2Lm11 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPITFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 26) |
| 2Lm12 | 2Hm | EIVLTQSPATLSLSPGERATLSCRASQSVSYMH |
| | | WYQQKPGQAPRLLIYATSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELS SLRSEDTAVYYCAR |
| | | SVYYSN.YWYFDLWGRGTLVTVSS |
| | | (SEQ ID NO: 27) |
| 2Lm13 | 2Hm | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWISNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | SVYYSN.YWYFDLWGRGTLVTVSS |
| | | (SEQ ID NO: 28) |
| 2Lm14 | 2Hm | EIVLTQSPATLSLSPGERATLSCRASQSVSYMH |
| | | WYQQKPGQAPRLLIYATSNLASGIPARFSGSGS |
| | | GTDFTLTISSLEPEDFAVYYCQQWISNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | SVYYSN.YWYFDLWGRGTLVTVSS |
| | | (SEQ ID NO: 29) |
| 2Lm15 | 2Hm | EIVLTQSPATLSLSPGERATLSCRASQSVSYIHW |
| | | YQQKPGQAPRLLIYAPSNLASGIPARFSGSGSG |
| | | TDFTLTISSLEPEDFAVYYCQQWISNPPTFGQG |
| | | TKVEIKDGGGSGGGGSGGGGSSQVQLVQSGA |
| | | EVKKPGASVKVSCKASGYTFTSYNMHWVRQ |
| | | APGQGLEWMGAIYPGNGDTSYNQKFKGRVT |
| | | MTRDTSTSTVYMELSSLRSEDTAVYYCAR |
| | | SVYYSN.YWYFDLWGRGTLVTVSS |
| | | (SEQ ID NO: 30) |
| 2Lm16 | 2Hm3 | EIVLTQSPATLSLSPGERATLSCRASSSVSYMH |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 31) |
| 2Lm17-3 | 2Hm3 | EIVLTQSPATLSLSPGERATLSCRASQSVSYLS |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCA |
| | | S,YYSNSYWYFDLWGRGTLVTVSS |
| | | (SEQ ID NO: 32) |
| 2Lm17-4 | 2Hm3 | EIVLTQSPATLSLSPGERATLSCRASQSVSYLT |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 33) |
| 2Lm17-6 | 2Hm3 | EIVLTQSPATLSLSPGERATLSCRASQSVSYLY |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | S.YYSNSYWYFDLWGRGTLVTVSS |
| | | (SEQ ID NO: 34) |
| 2Lm17-8 | 2Hm3 | EIVLTQSPATLSLSPGERATLSCRASQSVSYLH |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 35) |
| 2Lm17-12 | 2Hm3 | EIVLTQSPATLSLSPGERATLSCRASQSVSYLN |
| | | WYQQKPGQAPRLLIYAPSNLASGIPARFSGSG |
| | | SGTDFTLTISSLEPEDFAVYYCQQWSFNPPTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFK |
| | | GRVTMTRDTSTSTVYMELSSLRSEDTAVYYCA |
| | | RS.YYSNSYWYFDLWGRGTLVTVS S |
| | | (SEQ ID NO: 36) |
| 2Lm17-14 | 2Hm3 | EIVLTQSPATLSLSPGERATLSCRASQSVSYLA |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWVR |
| | | QAPGQGLEWMGAIYPGNGDTSYNQKFKGRVT |
| | | MTRDTSTSTVYMELSSLRSEDTAVYYCAR |
| | | S.YYSNSYWYFDLWGRGTLVTVSS |
| | | (SEQ ID NO: 37) |
| 2Lm18-2 | 2Hm3 | EIVLTQSPATLSLSPGERATLSCRASSSVSYLA |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 38) |
| 2Lm18-3 | 2Hm3 | EIVLTQSPATLSLSPGERATLSCRASSSVSYLN |
| | | WYQQKPGQAPRLLIYAPSNLASGIPARFSGS |
| | | GSGTDFTLTISSLEPEDFAVYYCQQWSFNPPT |
| | | FGQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCAR |
| | | S.YYSNSYWYFDLWGRGTLVTVSS |
| | | (SEQ ID NO: 39) |
| 2Lm18-4 | 2Hm3 | EIVLTQSPATLSLSPGERATLSCRASSSVSYLD |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 40) |
| 2Lm18-5 | 2Hm3 | EIVLTQSPATLSLSPGERATLSCRASSSVSYLSW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 41) |
| 2Lm18-14 | 2Hm3 | EIVLTQSPATLSLSPGERATLSCRASSSVSYLHW |
| | | YQQKPGQAPRLLIYAPSNLASGIPARFSGSGSGT |
| | | DFTLTISSLEPEDFAVYYCQQWSFNPPTFGQGTK |
| | | VEIKDGGGSGGGGSGGGGSSQVQLVQSGAEVKK |
| | | PGASVKVSCKASGYTFTSYNMHWVRQAPGQGL |
| | | EWMGAIYPGNGDTSYNQKFKGRVTMTRDTSTST |
| | | VYMELSSLRSEDTAVYYCAR |
| | | S.YYSNSYWYFDLWGRGTLVTVSS |
| | | (SEQ ID NO: 42) |
| 2Lm19-1 | 2Hm3 | EIVLTQSPATLSLSPGERATLSCRASQSVSYIDW |
| | | YQQKPGQAPRLLIYAPSNLASGIPARFSGSGSGT |
| | | DFTLTISSLEPEDFAVYYCQQWSFNPPTFGQGTK |
| | | VEIKDGGGSGGGGSGGGGSSQVQLVQSGAEVK |
| | | KPGASVKVSCKASGYTFTSYNMHWVRQAPGQG |
| | | LEWMGAIYPGNGDTSYNQKFKGRVTMTRDTST |
| | | STVYMELSSLRSEDTAVYYCAR |
| | | S.YYSNSYWYFDLWGRGTLVTVSS |
| | | (SEQ ID NO: 43) |
| 2Lm19-2 | 2Hm3 | EIVLTQSPATLSLSPGERATLSCRASQSVSYISW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 44) |
| 2Lm19-3 | 2Hm3 | EIVLTQSPATLSLSPGERATLSCRASQSVSYIVW |
| | | YQQKPGQAPRLLIYAPSNLASGIPARFSGSGSGT |
| | | DFTLTISSLEPEDFAVYYCQQWSFNPPTFGQGT |
| | | KVEIKDGGGSGGGGSGGGGSSQVQLVQSGAEV |
| | | KKPGASVKVSCKASGYTFTSYNMHWVRQAPG |
| | | QGLEWMGAIYPGNGDTSYNQKFKGRVTMTRD |
| | | TSTSTVYMELSSLRSEDTAVYYCAR |
| | | S.YYSNSYWYFDLWGRGTLVTVSS |
| | | (SEQ ID NO: 45) |
| 2Lm19-4 | 2Hm3 | EIVLTQSPATLSLSPGERATLSCRASQSVSYIAW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 46) |
| 2Lm19-7 | 2Hm3 | EIVLTQSPATLSLSPGERATLSCRASQSVSYITW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELS SLRSEDTAVYYCAR |
| | | S.YYSNSYWYFDLWGRGTLVTVSS |
| | | (SEQ ID NO: 47) |
| 2Lm19-9 | 2Hm3 | EIVLTQSPATLSLSPGERATLSCRASQSVSYIIW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 48) |
| 2Lm19-12 | 2Hm3 | EIVLTQSPATLSLSPGERATLSCRASQSVSYIPW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 49) |
| 2Lm19-14 | 2Hm3 | EIVLTQSPATLSLSPGERATLSCRASQSVSYINW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 50) |
| 2Lm20-1 | 2Hm3 | EIVLTQSPATLSLSPGERATLSCRASSSVSYISW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 51) |
| 2Lm20-2 | 2Hm3 | EIVLTQSPATLSLSPGERATLSCRASSSVSYIAW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 52) |
| 2Lm20-4 | 2Hm3 | EIVLTQSPATLSLSPGERATLSCRASSSVSYIVW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 53) |
| 2Lm20-8 | 2Hm3 | EIVLTQSPATLSLSPGERATLSCRASSSVNYIYW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 54) |
| 2Lm20-11 | 2Hm3 | EIVLTQSPATLSLSPGERATLSCRASSSVSYIDW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 55) |
| 2Lm20-12 | 2Hm3 | EIVLTQSPATLSLSPGERATLSCRASSSVSYIIW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 56) |
| 2Lm20-13 | 2Hm3 | EIVLTQSPATLSLSPGERATLSCRASSSVSYIYW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 57) |
| 2Lm5 | 2H5m3 | EIVLTQSPATLSLSPGERATLSCRASQSVSYMH |
| (18009) | | WYQQKPGQAPRLLIYAPSNLASGIPARFSGS |
| | | GSGTDFTLTISSLEPEDFAVYYCQQWSFNPPT |
| | | FGQGTKVEIKDGGGSGGGGSGGGGTGEVQLV |
| | | QSGAEVKKPGESLKISCKGSGYSFTSYNMHW |
| | | VRQMPGKGLEWMGAIYPGNGDTSYNQKFKG |
| | | QVTISADKSISTAYLQWSSLKASDTAMYYCA |
| | | RVVYYSNSYWYFDLWGRGTLVTVSS |
| | | (SEQ ID NO: 58) |
| 2Lm5 | 2H3m3 | EIVLTQSPATLSLSPGERATLSCRASQSVSYMH |
| (2Lm5 | | WYQQKPGQAPRLLIYAPSNLASGIPARFSGSGS |
| 2H3m3) | | GTDFTLTISSLEPEDFAVYYCQQWSFNPPTFG |
| | | QGTKVEIKDGGGSGGGGSGGGGTGEVQLLES |
| | | GGGLVQPGGSLRLSCAASGFTFSSYNMHWVR |
| | | QAPGKGLEWVSAIYPGNGDTSYNQKFKGRFT |
| | | ISRDNSKNTLYLQMNSLRAEDTAVYYCA |
| | | KSYYSNSYWYFDLWGRGTLVTVSS |
| | | (SEQ ID NO: 59) |
| IgG1 | Hinge | DQEPKSCDKTHTSPPSS |
| | CSSS | (SEQ ID NO: 60) |
| IgG1 | Hinge | DQEPKSCDKTHTCPPCP |
| | WT | (SEQ ID NO: 61) |
| IgG1 | Hinge | DQEPKSCDKTHTSPPCS |
| | LSCS | (SEQ ID NO: 62) |
| IgG1 | Hinge | DQEPKSSDKTHTCPPCS |
| | SCCS | (SEQ ID NO: 63) |
| IgG1 | Hinge | DQEPKSSDKTHTCPPCP |
| | SCOP | (SEQ ID NO: 64) |
| IgG1 | CH2CH | APELLGGPSVFLFPPKPKDTLMISRTPEVTCVVVDVSHEDPEVKF |
| | 3 | N |
| | WT | WYVDGVEVHNAKTKPREEQYNSTYRVVSVLTVLHQDWLNGKE |
| | | YKCKVSNKALPAPIEKTISKAKGQPREPQVYTLPPSRDELTKNQV |
| | | SLTCLVKGFYPSDIAVEWESNGQPENNYKTTPPVLDSDGSFFLYS |
| | | KLTVDKSRWQQGNVFSCSVMHEALHNHYTQKSLSLSPGK |
| | | (SEQ ID NO: 65) |
| IgG1 | CH2CH | APELLGGPSVFLFPPKPKDTLMISRTPEVTCVVVDVSHEDPEVKF |
| | 3 | N |
| | P331S | WYVDGVEVHNAKTKPREEQYNSTYRVVSVLTVLHQDWLNGKE |
| | | YKCKVSNKALPASIEKTISKAKGQPREPQVYTLPPSRDELTKNQV |
| | | SLTCLVKGFYPSDIAVEWESNGQPENNYKTTPPVLDSDGSFFLYS |
| | | KLTVDKSRWQQGNVFSCSVMHEALHNHYTQKSLSLSPGK |
| | | (SEQ ID NO: 66) |

Exemplary Full Length

| | |
|---|---|
| SEQ ID NO: 67 | EIVLTQSPATLSLSPGERATLSCRASQSVSYIVWYQQKPGQAPRL |
| | LIYAPSNLASGIPARFSGSGSGTDFTLTISSLEPEDFAVYYCQQWS |
| | FNPPTFGQGTKVEIKDGGGSGGGGSGGGGTGEVQLVQSGAEVK |
| | KPGESLKISCKGSGYSFTSYNMHWVRQMPGKGLEWMGAIYPGN |
| | GDTSYNQKFKGQVTISADKSISTAYLQWSSLKASDTAMYYCARS |
| | YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPASIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 68 | EIVLTQSPATLSLSPGERATLSCRASSSVSYIVWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | .YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPASIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 69 | QIVLSQSPAILSASPGEKVTMTCRASSSVSYMHWYQQKPGSSPKP |
| | WIYAPSNLASGVPARFSGSGSGTSYSLTISRVEAEDAATYYCQQ |
| | WSFNPPTFGAGTKLELKDGGGSGGGGSGGGGSSQAYLQQSGAE |
| | SVRPGASVKMSCKASGYTFTSYNMHWVKQTPRQGLEWIGAIYP |
| | GNGDTSYNQKFKGKATLTVDKSSSTAYMQLSSLTSEDSAVYFCA |
| | RVVYYSNSYWYFDVWGTGTTVTVSDQEPKSCDKTHTSPPCSAP |
| | ELLGGPSVFLFPPKPKDTLMISRTPEVTCVVVDVSHEDPEVKFNW |
| | YVDGVEVHNAKTKPREEQYNSTYRVVSVLTVLHQDWLNGKEY |
| | KCKVSNKALPAPIEKTISKAKGQPREPQVYTLPPSRDELTKNQVS |
| | LTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 70 | EIVLTQSPATLSLSPGERATLSCRASQSVSYIVWYQQKPGQAPRL |
| | LIYAPSNLASGIPARFSGSGSGTDFTLTISSLEPEDFAVYYCQQWS |
| | FNPPTFGQGTKVEIKDGGGSGGGGSGGGGTGEVQLVQSGAEVK |
| | KPGESLKISCKGSGYSFTSYNMHWVRQMPGKGLEWMGAIYPGN |
| | GDTSYNQKFKGQVTISADKSISTAYLQWSSLKASDTAMYYCARS |
| | YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 71 | EIVLTQSPATLSLSPGERATLSCRASSSVSYIVWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | .YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 72 | EIVLTQSPATLSLSPGERATLSCRASSSVSYIDWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | YYSNSYWYFDLWGRGTLVTVSSDQEPKSCDKTHTSPPSSAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 73 | EIVLTQSPATLSLSPGERATLSCRASSSVSYIVWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 74 | EIVLTQSPATLSLSPGERATLSCRASQSVSYIVWYQQKPGQAPRL |
| | LIYAPSNLASGIPARFSGSGSGTDFTLTISSLEPEDFAVYYCQQWS |
| | FNPPTFGQGTKVEIKDGGGSGGGGSGGGGTGEVQLVQSGAEVK |
| | KPGESLKISCKGSGYSFTSYNMHWVRQMPGKGLEWMGAIYPGN |
| | GDTSYNQKFKGQVTISADKSISTAYLQWSSLKASDTAMYYCARV |
| | VYYSNSYWYFDLWGRGTLVTVSSDQEPKSCDKTHTSPPCSAPEL |
| | LGGPSVFLFPPKPKDTLMISRTPEVTCVVVDVSHEDPEVKFNWY |
| | VDGVEVHNAKTKPREEQYNSTYRVVSVLTVLHQDWLNGKEYK |
| | CKVSNKALPASIEKTISKAKGQPREPQVYTLPPSRDELTKNQVSL |
| | TCLVKGFYPSDIAVEWESNGQPENNYKTTPPVLDSDGSFFLYSKL |
| | TVDKSRWQQGNVFSCSVMHEALHNHYTQKSLSLSPGK |
| SEQ ID NO: 75 | EIVLTQSPATLSLSPGERATLSCRASSSVSYMIWYQQKPGQAPRL |
| | LIYAISNLASGIPARFSGSGSGTDFTLTISSLEPEDFAVYYCQQWI |
| | SNPLTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | VYYSN.YWYFDLWGRGTLVTVSSDQEPKSCDKTHTCPPCPAPEL |
| | LGGPSVFLFPPKPKDTLMISRTPEVTCVVVDVSHEDPEVKFNWY |
| | VDGVEVHNAKTKPREEQYNSTYRVVSVLTVLHQDWLNGKEYK |
| | CKVSNKALPAPIEKTISKAKGQPREPQVYTLPPSRDELTKNQVSL |
| | TCLVKGFYPSDIAVEWESNGQPENNYKTTPPVLDSDGSFFLYSKL |
| | TVDKSRWQQGNVFSCSVMHEALHNHYTQKSLSLSPGK |
| SEQ ID NO: 76 | EIVLTQSPATLSLSPGERATLSCRASSSVSYIIWYQQKPGQAPRLLI |
| | YAPSNLASGIPARFSGSGSGTDFTLTISSLEPEDFAVYYCQQWSFN |
| | PPTFGQGTKVEIKDGGGSGGGGSGGGGSSQVQLVQSGAEVKKP |
| | GASVKVSCKASGYTFTSYNMHWVRQAPGQGLEWMGAIYPGNG |
| | DTSYNQKFKGRVTMTRDTSTSTVYMELSSLRSEDTAVYYCARSY |
| | YSNSYWYFDLWGRGTLVTVSSDQEPKSCDKTHTSPPSSAPELLG |
| | GPSVFLFPPKPKDTLMISRTPEVTCVVVDVSHEDPEVKFNWYVD |
| | GVEVHNAKTKPREEQYNSTYRVVSVLTVLHQDWLNGKEYKCK |
| | VSNKALPASIEKTISKAKGQPREPQVYTLPPSRDELTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGK |

### EQUIVALENTS

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### INCORPORATION OF REFERENCES

All publications and patent documents cited in this application are incorporated by reference in their entirety to the same extent as if the contents of each individual publication or patent document were incorporated herein.

What is claimed is:

1. A method of purifying a small modular immunopharmaceutical protein from a protein preparation containing high molecular weight aggregates comprising a step of subjecting the protein preparation to hydroxyapatite chromatography under an operating condition such that the purified small modular immunopharmaceutical protein contains less than 4% aggregates.

2. The method of claim 1, wherein the method comprises no more than 3 chromatography steps.

3. The method of claim 1, wherein the operating condition comprises eluting the small modular immunopharmaceutical protein from a hydroxyapatite chromatography column in a phosphate buffer.

4-5. (canceled)

6. The method of claim 3, wherein the phosphate buffer comprises sodium phosphate, potassium phosphate, and/or lithium phosphate.

7. The method of claim 3, wherein the phosphate buffer comprises sodium phosphate at a concentration ranging from 1 mM to 50 mM.

8. The method of claim 3, wherein the phosphate buffer further comprises sodium chloride at a concentration ranging from 100 mM to 2.5 M.

9. The method of claim 3, wherein the phosphate buffer comprises sodium phosphate at a concentration ranging from 2 mM to 32 mM and sodium chloride at a concentration ranging from 100 mM to 1.6 M.

10. (canceled)

11. The method of claim 1, wherein the operating condition comprises eluting the small modular immunopharmaceutical protein from a hydroxyapatite chromatography column by a NaCl gradient.

12. The method of claim 1, wherein the operating condition comprises eluting the small modular immunopharmaceutical protein from a hydroxyapatite chromatography column by a NaCl step elution method.

13. The method of claim 1, wherein the operating condition comprises eluting the small modular immunopharmaceutical protein from a hydroxyapatite chromatography column by a phosphate gradient.

14. The method of claim 13, wherein the phosphate gradient is a linear gradient.

15. The method of claim 13, wherein the phosphate gradient is a step gradient.

16-19. (canceled)

20. The method of claim 1, wherein the method further comprises a step of purifying the protein preparation by affinity chromatography before the hydroxyapatite chromatography.

21. The method of claim 20, wherein the affinity chromatography uses a protein absorbent that binds to a constant immunoglobulin domain.

22-23. (canceled)

24. The method of claim 21, wherein the protein absorbent comprises protein A.

25-33. (canceled)

34. The method of claim 20, wherein the method further comprises adding an additive to promote binding to sorbents.

35. The method of claim 1, wherein the method further comprises a step of purifying the protein preparation by anion exchange chromatography using an anion exchange chromatography resin.

36. The method of claim 20, wherein the method further comprises a step of purifying the protein preparation by anion exchange chromatography after the affinity chromatography but before the hydroxyapatite chromatography.

37. The method of claim 35, wherein the method further comprises a step of adding an additive to enhance binding of the small modular immunopharmaceutical protein and/or impurities to the anion exchange chromatography resin.

38. The method of claim 37, wherein the additive comprises a nonionic organic polymer.

39-40. (canceled)

41. The method of claim 1, the method further comprises one or more filtration steps.

42-43. (canceled)

44. The method of claim 1, wherein the method further comprises a step of adding an additive to induce protein precipitation of one or more contaminants from the protein preparation such that the small modular immunopharmaceutical protein can be further separated from contaminates.

45. The method of claim 44, wherein the additive comprises a nonionic organic polymer.

46-47. (canceled)

48. The method of claim 1, wherein the purified small modular immunopharmaceutical protein contains less than 2% aggregates.

49. (canceled)

50. The method of claim 1, wherein the protein preparation contains more than 10% high molecular weight aggregates.

51-53. (canceled)

54. The method of claim 1, wherein the protein preparation contains less than 30% high molecular weight aggregates.

55-58. (canceled)

59. The method of claim 1, wherein the small modular immunopharmaceutical protein binds specifically to CD20.

60. The method of claim 59, wherein the small modular immunopharmaceutical protein comprises an amino acid sequence having at least 80% identity to any one of SEQ ID NOs:1-59 and 67-76.

61. The method of claim 1, wherein the protein preparation is prepared from cultured bacterial cells, mammalian cells, insect cells, plant cells, yeast cells, cell-free medium, transgenic animals or plants.

62. The method of claim 1, wherein the protein preparation is a cell culture medium preparation.

63. The method of claim 62, wherein the culture medium preparation comprises the small modular immunopharmaceutical protein secreted from cultured cells.

64. The method of claim 63, wherein the cultured cells are CHO cells.

65. The method of claim 62, wherein the culture medium preparation is prepared from a large scale bioreactor.

66-67. (canceled)

68. A method of purifying a small modular immunopharmaceutical protein from a protein preparation containing high molecular weight aggregates, the method comprising subjecting the protein preparation to (a) affinity chromatography and/or ion exchange chromatography, and (b) hydroxyapatite chromatography under operating conditions such that the purified small modular immunopharmaceutical protein contains less than 4% aggregates.

69. The method of claim 68, wherein the protein preparation is subjected to (a1) affinity chromatography, (a2) ion exchange chromatography, and (b) hydroxyapatite chromatography.

70. The method of claim 68, wherein the protein preparation is subjected to (a1) cation exchange chromatography, (a2) anion exchange chromatography, and (b) hydroxyapatite chromatography.

71. The method of claim 68, wherein the method comprises no more than 3 chromatography steps.

72-78. (canceled)

79. The method of claim 69, wherein the affinity chromatography is MabSelect™ rProtein A affinity chromatography, the ion exchange chromatography is tentacle anion exchange chromatography, and the hydroxyapatite chromatography is Type I ceramic hydroxyapatite chromatography.

80-86. (canceled)

87. The method of claim 68, wherein the small modular immunopharmaceutical protein binds specifically to CD20.

88. The method of claim 87, wherein the small modular immunopharmaceutical protein comprises an amino acid sequence having at least 80% identity to SEQ ID NOs:1-59 and 67-76.

89. A small modular immunopharmaceutical protein purified using a method of claims 1.

90-98. (canceled)

99. A pharmaceutical composition comprising a small modular immunopharmaceutical protein and a pharmaceutically acceptable carrier, wherein the small modular immunopharmaceutical protein comprises less than 4% high molecular weight aggregates.

100-102. (canceled)


SEQUENCE LISTING (77 sequences)

SEQ ID NO: 1 - protein, 243 aa, Artificial Sequence
  SMIP 18011
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSS

SEQ ID NO: 2 - protein, 244 aa, Artificial Sequence
  SMIP 18008
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSS

SEQ ID NO: 3 - protein, 244 aa, Artificial Sequence
  SMIP 18010
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSS

SEQ ID NO: 4 - protein, 244 aa, Artificial Sequence; identical to SEQ ID NO: 3
  SMIP 18009

SEQ ID NO: 5 - protein, 244 aa, Artificial Sequence
  SMIP 2Lm5 2H3m3
EIVLTQSPATLSLSPGERATLSCRASQSVSSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCAKSYYSNSYWYFDLWGRGTLVTVSS

SEQ ID NO: 6 - protein, 244 aa, Artificial Sequence
  SMIP 2L 2Hm
EIVLTQSPATLSLSPGERATLSCRASSSVSSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 7 - protein, 243 aa, Artificial Sequence
  SMIP 2Lm 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 8 - protein, 244 aa, Artificial Sequence
  SMIP 2Lm 2H
EIVLTQSPATLSLSPGERATLSCRASSSVSYMIWYQQKPGQAPRLLIYAISNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDLWGRGTLVTVSS

SEQ ID NO: 9 - protein, 244 aa, Artificial Sequence
  SMIP 2Lm1 2Hm
EIVLTQSPATLSLSPGERATLSCRASQSSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 10 - protein, 245 aa, Artificial Sequence
  SMIP 2Lm1 2H
EIVLTQSPATLSLSPGERATLSCRASQSSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDLWGRGTLVTVSS

SEQ ID NO: 11 - protein, 243 aa, Artificial Sequence
  SMIP 2Lm2 2Hm
EIVLTQSPATLSLSPGERATLSCRASQSVSYMIWYQQKPGQAPRLLIYAISNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 12 - protein, 243 aa, Artificial Sequence
  SMIP 2Lm3 2Hm
EIVLTQSPATLSLSPGERATLSCRASSSVSYMIWYQQKPGQAPRLLIYAISNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 13 - protein, 244 aa, Artificial Sequence
  SMIP 2Lm4 2Hm
EIVLTQSPATLSLSPGERATLSCRASQSVSSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 14 - protein, 243 aa, Artificial Sequence
  SMIP 2Lm5 2Hm
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 15 - protein, 243 aa, Artificial Sequence
  SMIP 2Lm5-1 2Hm3
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 16 - protein, 243 aa, Artificial Sequence
  SMIP 2Lm5-2 2Hm4
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVYYSNSYWYFDLWGRGTLVTVSS

SEQ ID NO: 17 - protein, 243 aa, Artificial Sequence
  SMIP 2Lm5-3 2Hm5
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYNSYWYFDLWGRGTLVTVSS

SEQ ID NO: 18 - protein, 243 aa, Artificial Sequence
  SMIP 2Lm6 2Hm
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 19 - protein, 243 aa, Artificial Sequence
  SMIP 2Lm6-1 2Hm3
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 20 - protein, 243 aa, Artificial Sequence
  SMIP 2Lm6-2 2Hm4
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVYYSNSYWYFDLWGRGTLVTVSS

SEQ ID NO: 21 - protein, 243 aa, Artificial Sequence
  SMIP 2Lm6-3 2Hm5
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYNSYWYFDLWGRGTLVTVSS

SEQ ID NO: 22 - protein, 243 aa, Artificial Sequence
  SMIP 2Lm7 2Hm
EIVLTQSPATLSLSPGERATLSCRASSSVSYMHWYQQKPGQAPRLLIYATSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 23 - protein, 243 aa, Artificial Sequence
  SMIP 2Lm8 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPYTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 24 - protein, 243 aa, Artificial Sequence
  SMIP 2Lm9 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPFTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 25 - protein, 243 aa, Artificial Sequence
  SMIP 2Lm10 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPLTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 26 - protein, 243 aa, Artificial Sequence
  SMIP 2Lm11 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPITFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 27 - protein, 243 aa, Artificial Sequence
  SMIP 2Lm12 2Hm
EIVLTQSPATLSLSPGERATLSCRASQSVSYMHWYQQKPGQAPRLLIYATSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 28 - protein, 243 aa, Artificial Sequence
  SMIP 2Lm13 2Hm
EIVLTQSPATLSLSPGERATLSCRASQSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 29 - protein, 243 aa, Artificial Sequence
  SMIP 2Lm14 2Hm
EIVLTQSPATLSLSPGERATLSCRASQSVSYMHWYQQKPGQAPRLLIYATSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 30 - protein, 243 aa, Artificial Sequence
  SMIP 2Lm15 2Hm
EIVLTQSPATLSLSPGERATLSCRASQSVSYIHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 31 - protein, 243 aa, Artificial Sequence
  SMIP 2Lm16 2Hm3
EIVLTQSPATLSLSPGERATLSCRASSSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 32 - protein, 242 aa, Artificial Sequence
  SMIP 2Lm17-3 2Hm3
EIVLTQSPATLSLSPGERATLSCRASQSVSYLSWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCASYYSNSYWYFDLWGRGTLVTVSS

SEQ ID NO: 33 - protein, 486 aa, Artificial Sequence
  SMIP 2Lm17-4 2Hm3
EIVLTQSPATLSLSPGERATLSCRASQSVSYLTWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSEIVLTQSPATLSLSPGERATLSCRASQSVSYLTWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 34 - protein, 243 aa, Artificial Sequence
  SMIP 2Lm17-6 2Hm3
EIVLTQSPATLSLSPGERATLSCRASQSVSYLYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 35 - protein, 243 aa, Artificial Sequence
  SMIP 2Lm17-8 2Hm3
EIVLTQSPATLSLSPGERATLSCRASQSVSYLHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 36 - protein, 243 aa, Artificial Sequence
  SMIP 2Lm17-12 2Hm3
EIVLTQSPATLSLSPGERATLSCRASQSVSYLNWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 37 - protein, 243 aa, Artificial Sequence
  SMIP 2Lm17-14 2Hm3
EIVLTQSPATLSLSPGERATLSCRASQSVSYLAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 38 - protein, 243 aa, Artificial Sequence
  SMIP 2Lm18-2 2Hm3
EIVLTQSPATLSLSPGERATLSCRASSSVSYLAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 39 - protein, 243 aa, Artificial Sequence
  SMIP 2Lm18-3 2Hm3
EIVLTQSPATLSLSPGERATLSCRASSSVSYLNWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 40 - protein, 243 aa, Artificial Sequence
  SMIP 2Lm18-4 2Hm3
EIVLTQSPATLSLSPGERATLSCRASSSVSYLDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 41 - protein, 243 aa, Artificial Sequence
  SMIP 2Lm18-5 2Hm3
EIVLTQSPATLSLSPGERATLSCRASSSVSYLSWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 42 - protein, 243 aa, Artificial Sequence
  SMIP 2Lm18-14 2Hm3
EIVLTQSPATLSLSPGERATLSCRASSSVSYLHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 43 - protein, 243 aa, Artificial Sequence
  SMIP 2Lm19-1 2Hm3
EIVLTQSPATLSLSPGERATLSCRASQSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 44 - protein, 243 aa, Artificial Sequence
  SMIP 2Lm19-2 2Hm3
EIVLTQSPATLSLSPGERATLSCRASQSVSYISWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 45 - protein, 243 aa, Artificial Sequence
  SMIP 2Lm19-3 2Hm3
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 46 - protein, 243 aa, Artificial Sequence
  SMIP 2Lm19-4 2Hm3
EIVLTQSPATLSLSPGERATLSCRASQSVSYIAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 47 - protein, 243 aa, Artificial Sequence
  SMIP 2Lm19-7 2Hm3
EIVLTQSPATLSLSPGERATLSCRASQSVSYITWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 48 - protein, 243 aa, Artificial Sequence
  SMIP 2Lm19-9 2Hm3
EIVLTQSPATLSLSPGERATLSCRASQSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 49 - protein, 243 aa, Artificial Sequence
  SMIP 2Lm19-12 2Hm3
EIVLTQSPATLSLSPGERATLSCRASQSVSYIPWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 50 - protein, 243 aa, Artificial Sequence
  SMIP 2Lm19-14 2Hm3
EIVLTQSPATLSLSPGERATLSCRASQSVSYINWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 51 - protein, 243 aa, Artificial Sequence
  SMIP 2Lm20-1 2Hm3
EIVLTQSPATLSLSPGERATLSCRASSSVSYISWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 52 - protein, 243 aa, Artificial Sequence
  SMIP 2Lm20-2 2Hm3
EIVLTQSPATLSLSPGERATLSCRASSSVSYIAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 53 - protein, 243 aa, Artificial Sequence
  SMIP 2Lm20-4 2Hm3
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 54 - protein, 243 aa, Artificial Sequence
  SMIP 2Lm20-8 2Hm3
EIVLTQSPATLSLSPGERATLSCRASSSVNYIYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 55 - protein, 243 aa, Artificial Sequence
  SMIP 2Lm20-11 2Hm3
EIVLTQSPATLSLSPGERATLSCRASSSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 56 - protein, 243 aa, Artificial Sequence
  SMIP 2Lm20-12 2Hm3
EIVLTQSPATLSLSPGERATLSCRASSSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 57 - protein, 243 aa, Artificial Sequence
  SMIP 2Lm20-13 2Hm3
EIVLTQSPATLSLSPGERATLSCRASSSVSYIYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 58 - protein, 244 aa, Artificial Sequence; identical to SEQ ID NO: 2
  SMIP 2Lm5 2H5m3

SEQ ID NO: 59 - protein, 243 aa, Artificial Sequence
  SMIP 2Lm5 2H3m3
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCAKSYYSNSYWYFDLWGRGTLVTVSS

SEQ ID NO: 60 - protein, 17 aa, Artificial Sequence
  Hinge CSSS
DQEPKSCDKTHTSPPSS

SEQ ID NO: 61 - protein, 17 aa, Artificial Sequence
  Hinge WT
DQEPKSCDKTHTCPPCP

SEQ ID NO: 62 - protein, 17 aa, Artificial Sequence
  Hinge CSCS
DQEPKSCDKTHTSPPCS

SEQ ID NO: 63 - protein, 17 aa, Artificial Sequence
  Hinge SCCS
DQEPKSSDKTHTCPPCS

SEQ ID NO: 64 - protein, 17 aa, Artificial Sequence
  Hinge SCCP
DQEPKSSDKTHTCPPCP

SEQ ID NO: 65 - protein, 217 aa, Artificial Sequence
  CH2 CH3 WT
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 217 aa, Artificial Sequence
  CH2 CH3 P331S
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 477 aa, Artificial Sequence
  Exemplary full lenth SMIP
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 477 aa, Artificial Sequence
  Exemplary full length SMIP
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 477 aa, Artificial Sequence
  Exemplary full length SMIP
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKDGGGSGGGGSGGGGSSQAYLQQSGAESVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 477 aa, Artificial Sequence
  Exemplary full length SMIP
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 477 aa, Artificial Sequence
  Exemplary full length SMIP
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 477 aa, Artificial Sequence
  Exemplary full length SMIP
EIVLTQSPATLSLSPGERATLSCRASSSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSCDKTHTSPPSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 477 aa, Artificial Sequence; identical to SEQ ID NO: 71
  Exemplary full length SMIP

SEQ ID NO: 74 - protein, 478 aa, Artificial Sequence
  Exemplary full legth SMIP
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 477 aa, Artificial Sequence
  Exemplary full length SMIP
EIVLTQSPATLSLSPGERATLSCRASSSVSYMIWYQQKPGQAPRLLIYAISNLASGIPARFSGSGSGTDFTLTISSLEPEDFAVYYCQQWISNPLTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSSDQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 477 aa, Artificial Sequence
  Exemplary full length SMIP
EIVLTQSPATLSLSPGERATLSCRASSSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSCDKTHTSPPSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 12 aa, Artificial Sequence
  Flexible linker
GGGSGGGGSGGS